(19) United States
(12) Patent Application Publication
(10) Pub. No.: US 2026/0216459 A1
(43) Pub. Date: Jul. 30, 2026

(54) SMART MEDICAL DEVICE MANAGEMENT

(71) Applicant: Stryker Corporation, Portage, MI (US)

(72) Inventors: Darren Schaaf, Portage, MI (US); Adithya Chandrashekharan, Portage, MI (US); Kerry Castor, Kalamazoo, MI (US); Scott Batzer, Vicksburg, MI (US); Aaron Furman, Kalamazoo, MI (US); Habib Imad Baydoun, Chelsea, MI (US); John Daniel Zender, Woodinville, WA (US); Michael John Baran, Kalamazoo, MI (US)

(73) Assignee: Stryker Corporation, Portage, MI (US)

(21) Appl. No.: 19/141,996

(22) PCT Filed: Dec. 20, 2023
(86) PCT No.: PCT/US2023/085182

§ 371 Date: Jun. 20, 2025

Related U.S. Application Data
(60) Provisional application No. 63/476,253, filed on Dec. 20, 2022.

(51) Int. Cl. A61M 16/06; A61M 16/00; A61M 16/08; A61M 16/16

(52) U.S. Cl. CPC A61M 16/06; A61M 16/0084; A61M 16/024; A61M 16/0858; A61M 16/161; A61M 2016/0039; A61M 2205/13; A61M 2205/15; A61M 2205/18; A61M 2205/505; A61M 2230/432; A61M 2230/50; A61M 2230/63

(57) ABSTRACT

There is described a medical device (100), comprising:—an airway adaptor (212, 312) configured to be connected to an airway of a patient;—a first sensor (104, 202, 302) a configured to detect a first parameter of a space fluidly connected to the airway adaptor, the first parameter being for example an air flow rate, an air pressure, an O2 level, a CO2 level, or a humidity level;—a second sensor (104, 204, 304) configured to detect a second parameter of the space, the second parameter being for example an O2 level or an air pressure;—a display (216, 316); and-a processor (510) configured to determine a relationship between the first parameter and the second parameter, based on determining that the relationship has changed over multiple ventilations, identify a leak between the airway adaptor and the airway of the patient, generate an alert indicating the leak, and cause the display to output the alert. The medical device may comprise for example a bag-valve-mask (BVM) and the airway adaptor may comprise for example a mask, a supraglottic device, or an endotracheal tube.

### CROSS-REFERENCE TO RELATED APPLICATIONS

This application is the 35 U.S.C. § 371 National Phase of International App. No. PCT/US 2023/085182, filed on Dec. 20, 2023, which claims priority to U.S. Provisional App. No. 63/476,253, titled “Smart Medical Device Management” and filed on Dec. 20, 2022, each of which is incorporated by reference herein in its entirety.

### BACKGROUND

Medical devices including bag-valve-mask (BVM) devices can be used to facilitate patient monitoring, facilitate patient treatments, or a combination of both. Care providers can use the BVM devices in emergency settings when patients are not breathing. The BVM devices can be used to provide positive pressure ventilation to the patients. The care providers can place masks of the BVM devices onto faces of the patients and contract the bags of the BVM devices to force oxygenated air into the respiratory tracts of the patients. The BVM devices are frequently used in out-of-hospital settings, emergency rooms, or critical care settings where mechanical ventilation devices are unavailable.

### BRIEF DESCRIPTION OF THE DRAWINGS

FIG. 1 illustrates an example illustration of a medical device being interconnected with a monitoring device and including one or more sensors.

FIG. 2 illustrates an example illustration of a medical device including an air flow sensor and an air pressure sensor.

FIG. 3 illustrates an example illustration of a medical device including one or more modular sensors.

FIG. 4 illustrates an example illustration of a medical device including an air flow sensor, CO2 sensor, and an air pressure sensor.

FIG. 5 illustrates an example illustration of a medical device and a modular sensor, and an example schematic diagram of an electrical circuit of the modular sensor.

FIGS. 6A and 6B illustrate example illustrations of a medical device being operated to perform ventilation.

FIG. 7 illustrates an example environment for utilizing a medical device including at least one sensor to treat a patient at a rescue scene.

FIG. 8 illustrates an example signal flow for exchanging communications between a medical device, a monitoring device, and a treating device.

FIG. 9 illustrates an example environment of a medical device including one or more sensors and being interconnected with a monitoring device and with an intermediary device.

FIG. 10 illustrates an example process for managing a medical device including an air flow sensor and an air pressure sensor.

FIG. 11 illustrates an example process for managing a medical device including one or more modular sensors.

FIG. 12 illustrates an example process for managing a medical device including an air flow sensor, CO2 sensor, and an air pressure sensor.

### DETAILED DESCRIPTION

In some environments, the BVM devices can include sensors utilized to monitor conditions of patients. The conditions of the patients can be monitored based on different types of physiological parameters detected by different types of sensors. Various implementations described herein relate to techniques for utilizing sensors of bag-valve-mask (BVM) devices to identify leaks. For instance, a BVM device detects a physiological parameter and determines whether a leak is present between the medical device and a face of a patient in response to the physiological parameter. In particular examples, BVM devices include sensors to detect flow rates of air in spaces of the BVM devices. In those or other examples, the BVM devices determine whether leaks are present between the medical devices and faces of patients in response to the flow rates.

However, particular environments may be chaotic, noisy, and/or visibly obscured. It is therefore important that a given medical device (e.g., a BVM device) being utilized to facilitate patient monitoring and/or to administer medical treatment include components enabling effective utilization of the medical device according to a variety of environmental, patient, and/or caregiver conditions. In addition, in high-stress clinical and/or field environments, a user may experience difficulty in maintaining a seal between the medical device and a face of a patient and/or in ensuring that the seal is being maintained. In addition, a user may experience difficulty in accurately measuring a flow rate of air in the medical device. It is therefore preferred, in some environments, that medical devices determine a variety of types of medical device data enabling operation of the medical devices with minimal or no interruption, difficulty, and/or uncertainty.

According to various implementations of the present disclosure, a medical device (e.g., a BVM device) determines medical device data. The medical device data includes air flow and air pressure data determined by sensors, physiological parameter data determined by modular sensors, and/or aggregated data determined by multiple sensors. One or more portions of the medical device data is communicated to external devices and/or being utilized to output video or audio coaching data. The medical device includes a user interface (UI) to output data and a transceiver to exchange communications with other devices.

The medical device includes sensors utilized to detect physiological parameters, such as a flow rate (or “air flow”) of air and a pressure (e.g., an air pressure). The physiological parameters include artifacts that are indicative of treatment (e.g., assisted ventilations) being administered to a patient by the medical device. The physiological parameters are detected in a space that is fluidly connected to an airway of the patient. For instance, the treatment includes the ventilations being administered by a user contracting a bag of the medical device. The medical device determines ventilation volumes by analyzing the air flow during the ventilations. The medical device identifies a ratio between air pressures and the ventilation volumes associated with the ventilations. The medical device includes other sensors to detect other physiological parameters, the other physiological parameters including a CO₂ level, an O₂ level, a humidity, a temperature, and a mask-to-face contact level. The medical device determines whether a discrepancy is present between different ones of the ratios and determine, in response to the discrepancy and/or the other physiological parameters, whether a leak is present based on the discrepancy. The medical device outputs an alert in response to the leak being determined. Thus, implementations of the present disclosure enable the medical device to determine the discrepancy to accurately determine whether the seal is being maintained between the medical device and the patient.

Although the sensors may be utilized to detect the physiological parameters, as discussed above in the current disclosure, implementations are not so limited. In various examples, any of the physiological parameters may be communicated from the sensors, and/or from one or more devices including the sensors, which are communicatively connected to implementations of the medical device described herein.

According to those or other implementations of the present disclosure, the medical device (e.g., the BVM device) includes a modular sensor utilized to detect a physiological parameter. The modular sensor being coupled to the medical device is temporarily or permanently coupled between a bag of the medical device and a mask of the medical device. The modular sensor detects a physiological parameter in a space of the medical device that is fluidly connected to an airway of a patient. In some examples, the modular sensor is coupled to the medical device along with another modular sensor, which is utilized to detect another physiological parameter in the space. The physiological parameter is utilized, along with the other physiological parameter, to determine whether a leak is present. The medical device outputs an alert in response to the leak being determined. Thus, implementations of the present disclosure enable the medical device, which includes the modular sensors, to accurately determine whether the seal is being maintained between the medical device and the patient.

According to those or other implementations of the present disclosure, the medical device (e.g., the BVM device) includes sensors, a user interface (UI) (e.g., a display, an indicator, a speaker, a haptic feedback device, or a combination thereof), and a transceiver. The sensors are utilized to detect physiological parameters. The UI and the transceiver are controlled based on the physiological parameters being detected. The medical device includes a processor to determine a metric, which includes an air flow and air pressure ratio. The physiological parameters include an air flow, a CO₂ level, and an air pressure. The processor determines other metrics, the other metrics including a volume and air pressure ratio, an inspired and expired volume difference, a patient side respiration rate and provider side ventilation rate difference, a magnitude of a leak, and an end-tidal carbon dioxide. In response to the physiological parameters, the other physiological parameters, the metric, and/or the other metrics, the medical device determines whether a leak is present in a space of the medical device that is fluidly connected to an airway of a patient. Additionally or alternatively, the medical device determines coaching data, output visual and/or audio data, and/or exchange communications. Thus, implementations of the present disclosure enable the medical device, which detects the physiological parameters and/or the other physiological parameters and/or to determine the metric and/or the other metrics, to accurately determine whether the seal is being maintained between the medical device and the patient.

Implementations of the present disclosure are directed to improvements in the technical field of medical devices, including BVM devices. Rather than requiring complex configurations, assemblies, and interconnections of multiple devices, implementations described herein enable simplified and automated physiological parameter detecting and metric determining. In some cases, the medical devices detect various types of physiological parameters by various types of sensors, which are customisedly included in the medical devices, enabling the medical devices to be utilized in medical settings, particularly in emergency settings requiring rapid and accurate responses to complex and/or unexpected patient conditions.

Various examples will now be described with reference to the accompanying drawings.

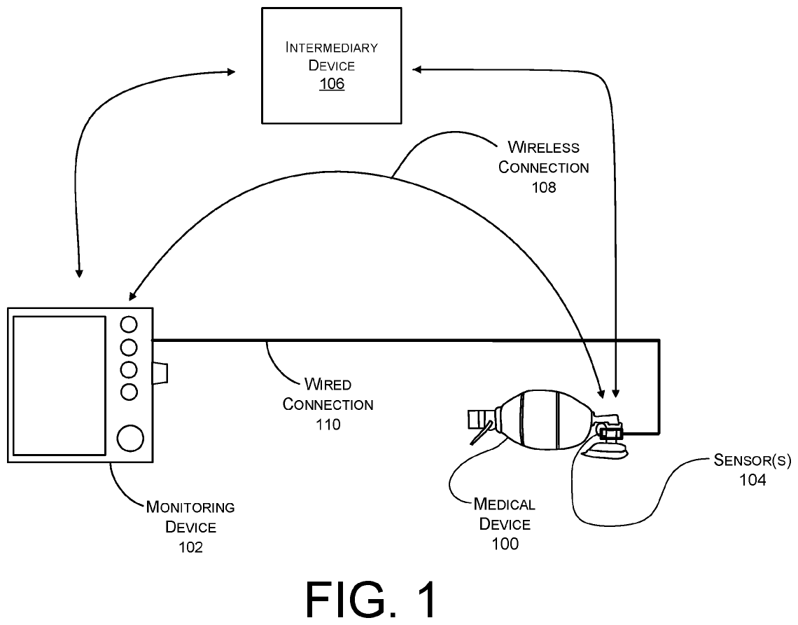

FIG. 1 illustrates an example illustration of a medical device 100 being interconnected with a monitoring device 102 and including one or more sensors 104. In some cases, the medical device 100 is a ventilation device. For instance, the medical device 100 includes a bag-valve-mask (BVM) device including a bag, a valve, and a mask. In such instance or another instance, the mask includes at least one of a cover, a cushion (e.g., an inflatable air cushion), an air bladder, a pillow, a mat, or the like, around a perimeter of an end of the mask that includes an opening. In such instance or another instance, the bag may include an air bladder.

In various implementations, the medical device 100 includes a processor. The processor, for example, performs any operations utilized to control and/or operate the medical device 100.

The mask forms a seal between the medical device 100 and a face of a patient to create a space that is fluidly connected to an airway of the patient. The medical device 100 includes the space, which extends from the environment and to the patient. The bag is contracted by a user for ventilation of air through the medical device 100.

In some cases, the sensor(s) 104 detect at least one physiological parameter. Examples of the sensor(s) 104, for instance, include an air flow sensor, an air pressure sensor, a CO₂ sensor, an O₂ sensor, a humidity sensor, a temperature sensor, or the like. Examples of the physiological parameter(s) include, for instance, a flow rate, an air pressure, a CO₂ level, an O₂ level, a humidity, a temperature, or the like.

In some examples, the medical device 100 detects the physiological parameter(s) and/or determines at least one metric to determine whether a leak is present in the space of the medical device that is fluidly connected to the airway of the patient. The medical device 100 detects the flow rate and the air pressure in the space. The medical device 100 determines ventilation volumes by analyzing the flow of air during ventilations when the bag is contracted and released. As used herein, the term “ventilation volume,” and its equivalents, refers to a volume of air that enters an airway of a patient, a volume of air that flows through a ventilation device (e.g., a BVM device), a volume of air that leaves the ventilation device, or any combination thereof. The medical device 100 identifies ratios between the ventilation volumes and pressures during the ventilations to determine discrepancies between the ratios.

Although various types of physiological parameters are detected by the sensor(s) 104, as discussed above in the current disclosure, it is not limited as such. In some examples, the parameter(s) include at least one of an electrocardiogram (ECG) of the patient, a temperature of the patient, a blood pressure of the patient, a blood oxygenation (e.g., a regional oxygenation (rSO₂, an arterial oxygen saturation, pulse oxygenation (SpO₂), etc.) of the patient, a heart rate of the patient, a pulse rate of the patient, or one or more airway parameters. As used herein, the term “airway parameter,” and its equivalents, may refer to a metric related to and/or indicative of air present in and/or flowing through lungs, a trachea, a throat, or mouth of a patient. Examples of airway parameters, which may be included among the physiological parameter(s), include parameters indicative of a characteristic of the airway of the patient, or a characteristic of the respirations of the patient, or a characteristic of ventilations being provided to the patient, such as airway pressure (e.g., positive end-expiratory pressure (PEEP), plateau pressure, peak inspiratory pressure (PIP), etc.), airway flow, airway volume (e.g., inspiratory volume, expiratory volume, tidal volume, or minute volume), respiration/ventilation rate, inspiratory and/or expiratory time, partial pressure of CO₂, end tidal CO₂, partial pressure of O₂, end tidal O₂, and so on.

In various examples, the medical device 100 communicates with an oximeter device (not illustrated), such as a pulse oximeter configured to detect SpO₂ of the patient. For instance, the medical device 100 receives, from the pulse oximeter, data indicative of a blood oxygenation of the patient. Based on the blood oxygenation, the medical device 100 may confirm whether ventilation is sufficient, accurate, and/or whether there is a leak in a seal between the medical device 100 and the face of the patient. For instance, the medical device 100 determines whether assisted ventilation performed on the patient is adequate by analyzing airway parameters of the patient, and can also confirm whether the ventilation is adequate by determining whether the blood oxygenation of the patient is sufficient (e.g., whether the oxygenation is greater than a threshold). In some cases, the medical device 100 infers or confirms that a leak is present based, at least in part, on the oxygenation of the patient being less than a threshold. In those or other cases, for example, the blood oxygenation is utilized to determine whether oxygenated air is being delivered to the patient (e.g., whether air being delivered includes oxygenated air above a threshold percentage, level, and/or amount), and/or whether the chest of the patient rises during inhalation beyond a threshold level, which may indicate whether the leak is present.

Although the various types of physiological parameters are utilized to determine whether the leak is present, as discussed above in the current disclosure, the present disclosure is not limited as such. In various cases, any of the physiological parameter(s), individually or in combination, such as the parameter(s) from the at least one pressure sensor within the cover (e.g., the air bladder) of the mask, the at least one capacitive sensor in, or on the surface of, the mask, the at least one resistive sensor in, or on the surface of, the mask, the at least one pressure sensor in, or on the surface of, the mask, the chest compression altitude sensor, and so on, may be utilized to determine whether the leak is present for purposes of implementing any of the techniques as discussed herein. In those or other cases, any of the physiological parameter(s), individually or in combination, such as the parameter(s) from the at least one pressure sensor, the at least one capacitive sensor, the at least one resistive sensor, the at least one pressure sensor, the chest compression altitude sensor, and so on, for example, may be utilized, alternatively or additionally to the physiological parameter(s) from any other sensors, such as the sensor(s) 104, to determine whether the leak is present for purposes of implementing any of the techniques as discussed herein.

Although the medical device 100 may include the BVM device, as discussed above in the current disclosure, it is not limited as such. In some examples, the medical device 100 includes a gas source, an element configured to move the gas in and out of the airway of the patient, as well as an airway adapter configured to connect a ventilation device to the airway of the patient. For instance, the gas source may include an O₂ tank or other container for an O2-containing gas that is administered to the patient. The element configured to move the gas in and out of the airway of the patient, in some cases, includes a manual device such as the BVM device (e.g., a bag-valve device, such as a bag that is manually squeezed by a rescuer in order to propel the gas into the airway of the patient and that is manually released by the rescuer in order to allow exhalation from the patient) or an automated device such as a mechanical ventilator. The medical device 100, in various cases, includes at least one duct (e.g., a tube, pipe, or the like) that is fluidly connected to the airway of the patient, and through which air is moved into, through, and out of the airway of the patient. In some cases, the medical device 100 includes a BVM that includes a number of valves, including a valve configured to prevent exhaled air from flowing back into the bag, thus avoiding rebreathing of expired CO2. A supraglottic device, in various examples, includes a bag attached to a duct that fits over or otherwise directs air to the glottic opening of the patient, without being inserted through the vocal cords. In various examples, the medical device 100 includes a valve that selectively vents a fluid circuit connecting an interior of the medical device 100 and the airway of the patient to an external environment.

In various cases, the medical device 100 includes an airway adapter (also referred to as an “airway interface”). In some implementations, the airway adapter includes a mask that is disposed on the face of the patient (e.g., over the mouth and nose of the patient), an endotracheal tube disposed in the trachea of the patient, or a supraglottic device disposed in a pharynx of the patient.

In some examples, the sensor(s) 104 of the medical device 100 include at least one modular sensor. Examples of the modular sensor(s) include a removable sensor, a detachable sensor, a temporarily connectable sensor, or the like. For instance, a modular sensor includes at least one thread, at least one rivet, at least one clasp, at least one tong, at least one fastener, at least one hook, at least one buckle, at least one pin, at least one screw, or the like. In such an instance or other instances, the modular sensor(s) are malleable and/or adjustable to connect the modular sensor(s) to the medical device 100 by pressure fitting and/or compression fitting the modular sensor(s) to the medical device 100. The medical device 100 determines whether a leak is present in the space of the medical device in response to the physiological parameter(s) detected by the modular sensor(s).

In some examples, the physiological parameter(s) detected by the medical device 100 include the flow rate, the CO₂ level, and the air pressure. The medical device 100 determines at least one metric in response to the flow rate, the CO₂ level, and the air pressure. Examples of the metric(s) include a volume and air pressure ratio, an inspired and expired volume difference, a patient side respiration rate and provider side ventilation rate difference, a magnitude of a leak, and an end-tidal carbon dioxide. As used herein, the term “inspired volume,” and its equivalents, can refer to a volume of air that enters an airway of a patient and/or a volume of air that leaves a ventilation device. As used herein, the term “expired volume,” and its equivalents, can refer to a volume of air that leaves an airway of a patient and/or a volume of air that enters a ventilation device from the airway of the patient. The medical device 100 determines that a leak is present between the face and the mask based on the flow rate, the CO₂ level, the air pressure, and the metric(s). The medical device 100 transmits, via the transceiver, a signal indicating the leak, and outputs, via the display, an alert indicating the leak.

In various implementations, the leak is detected by analyzing at least one of the physiological parameter(s) of the patient. The leak, for example, is detected based on any of the physiological parameter(s) being outside of a predetermined physiological parameter range. In some example, the predetermined physiological parameter range is determined based on an patient data, including an age, a weight, or a height, and so on, or any combination thereof, of the patient. The patient data is received from the monitoring device 102, and/or received via input by the user.

In some examples, a physiological parameter (e.g., a first parameter) of the space in the bag-valve mask that is fluidly connected to the airway of the patient and that is disposed on the face of the patient is detected. In various examples, a physiological parameter (e.g., a second parameter) of the space is detected. A relationship, for example, is determined between the first parameter and the second parameter. In some implementations, the relationship having changed over multiple ventilations performed on the patient by the bag-valve mask is determined. Based on determining that the relationship has changed over the multiple ventilations, for example, is used to identify a leak between the bag-valve mask and the face of the patient. In some cases, an alert indicating the leak is output.

In some examples, the first physiological parameter includes the air flow rate, the second physiological parameter includes the air pressure level or the O₂ level. An incongruity, for example, is identified based on the relationship initially being an analogous relationship, and further based on the relationship then having changed over the multiple ventilations to not be the analogous relationship.

In some examples, the first physiological parameter includes the CO₂ level, the second physiological parameter includes the O₂ level. An incongruity, for example, is identified based on the relationship initially being an inverse relationship, and further based on the relationship then having changed over the multiple ventilations to not be the inverse relationship.

In some examples, the first physiological parameter includes the CO₂ level, the second physiological parameter includes the humidity or the temperature. An incongruity, for example, is identified based on the relationship initially being an inverse relationship, and further based on the relationship then having changed over the multiple ventilations to not be the inverse relationship.

In some cases, determining whether the leak is present is based, exclusively, on the air pressure. For instance, the leak can be detected by determining that the air pressure in the medical device 100 is below a predetermined threshold. The leak, for instance, would lower the air pressure by venting the oxygenated air into the atmosphere during an inspiratory phase, or cause the medical device 100 to obtain air from the atmosphere during an expiratory phase. In some implementations, the leak is detected based on a waveform of the air pressure over time. For example, if the medical device 100 is appropriately sealed to the face of the patient, then the air pressure over time during the inspiratory phase would increase, because the pressure exerted by the lungs of the patient would increase as the lungs fill with air. Thus, the leak can be detected if the rate-of-change of the air pressure over time is below a threshold during the inspiratory phase.

In some cases, determining whether the leak is present is based on a ratio (e.g., a flow rate and air pressure ratio) between the flow rate and the air pressure. In various cases, determining whether the leak is present is based on a ratio (e.g., the volume and air pressure ratio) between a ventilation volume and the air pressure. For instance, if the medical device 100 is sealed to the face of the patient, then the flow rate may be expected to be positively correlated with the rate-of-change of the air pressure with respect to time. That is, if the medical device 100 is appropriately sealed to the face of the patient, the lungs of the patient would be expected to exert a greater pressure as the volume of air in the lungs increases. In contrast, if there is a leak present between the medical device 100 and the face of the patient, then the observed pressure in the medical device 100 would not change as the volume of the air that moves in and/or out of the medical device 100 changes. In some examples, the leak is detected by comparing a ratio of the flow rate and the derivative of the air pressure with respect to time to a threshold and/or by comparing a ratio of an integral of the flow rate with respect to time and the air pressure.

In some cases, determining whether the leak is present is based on a combination of the ventilation volume and at least one of the air pressure, the flow rate and air pressure ratio, or the volume and air pressure ratio. In some cases, determining whether the leak is present is based on the humidity or the temperature in the space that is fluidly connected to an airway of the patient. For example, the humidity or the temperature in the airway of the patient is utilized to determine whether the leak is present. In various cases, determining whether the leak is present based on a rate (e.g., a respiration rate) of inspired air minus a rate (e.g., a ventilation rate) of expired air.

In various examples, the respiration rate of inspired air being less than ventilation rate of expired air indicates a lack of a complete seal between the mask and the face. A difference determined based on the respiration rate minus the ventilation rate being less than a threshold difference, for example, indicates the leak. Air not flowing into the lungs of the patient during expiration based on the leak, for example, results in a lower amount of air flowing into the mask and from the lungs of the patient during inspiration.

In some examples, determining the leak is based on a compression level of the bag of the medical device being greater than or equal to a threshold compression level, and the air pressure level being less than the predetermined air pressure level. In some examples, determining the leak is based on a physiological characteristic at a time associated with a compression level of an air bag of the medical device being greater than or equal to a threshold compression level, the physiological characteristic representing the humidity level being less than the predetermined humidity level.

In some examples, determining whether the leak is present includes determining a result of a mask seal rating algorithm. A metric including the result of the mask seal rating algorithm is a value corresponding to a likelihood of the leak being present and impacting a patient health. The mask seal rating algorithm mask seal rating algorithm includes performing a tiered analysis. A first tier of the mask seal rating algorithm includes, for example, calculating a metric that includes a ratio of a ventilation volume to an air pressure based on a current breath; and calculating a metric that includes a ratio of a ventilation volume to an air pressure based on individual ones of at least one previous breath. A metric that includes a volume to pressure average, for example, is determined. Determining the volume to pressure average, for example, includes averaging a ratio (e.g., a volume to pressure ratio) of the ventilation volume to the air pressure based on the current breath and individual ones of the previous breath(s).

As used herein, the term “breath,” and its equivalents, can refer to an expiration, which includes air flowing into the lungs of the patient. As used herein, the term “volume,” and its equivalents, can refer to a volume of air entering the lungs of the patient during a breath. As used herein, the term “pressure,” and its equivalents, can refer to a level of pressure in the lungs of the patient during a breath

The determined volume to pressure ratio average being less than a threshold (e.g., a first threshold volume to pressure ratio average) may indicate, for example, a volume of air being relatively lower based on a portion of air flowing out of a separation between the mask and the face of the patient. The determined volume to pressure ratio average being greater than a threshold (e.g., a second threshold volume to pressure ratio average) may indicate, for example, a pressure of air being relatively lower based on a portion of air flowing out of a separation between the mask and the face of the patient. The determined volume to pressure ratio average being between the first threshold volume to pressure ratio average and the second threshold volume to pressure ratio average may indicate, for example, an absence of a leak between the mask and the face of the patient.

In some examples, an analogous relationship between the volume and the pressure changes indicates the pressure utilized by the lungs during inspiration, and the volume of the air during inspiration, are aligned with expected pressure and volume changes. The volume or the pressure changing inversely, for example, between ventilations may be used to identify the leak being present. In various examples, determining an inverse relationship between changes in the volume and pressure based on the pressure increasing and the volume decreasing is used to identify the leak.

A second tier of the mask seal rating algorithm includes, for example, calculating a metric that includes a difference (e.g., inspired volume versus expired volume difference) between an inspired volume versus an expired volume. The inspired volume versus expired volume difference being greater than a threshold inspired volume versus expired volume difference (e.g., a first threshold inspired volume versus expired volume difference), for example, may indicate the leak is present and relatively greater. The inspired volume versus expired volume difference being less than a threshold inspired volume versus expired volume difference (e.g., a second threshold inspired volume versus expired volume difference), for example, may indicate the leak is present and relatively smaller.

A third tier of the mask seal rating algorithm includes, for example, calculating a metric that includes a capnograph (e.g., EtCO₂) (or “end-tidal carbon dioxide level”) to determine whether to output the alert indicating the leak. The capnograph may be calculated, for example, based on the inspired volume versus expired volume difference being less than the second threshold inspired volume versus expired volume difference. The capnograph being relatively lower (e.g., 10 millimeters of mercury (mmHg), 5 mmHg, etc.) may indicate, for example, that the leak is having a relatively larger effect on a health of the patient. The capnograph being relatively higher (e.g., 35 mmHg, 40 mmHg, 45 mmHg, etc.) may indicate, for example, that the leak is having a relatively smaller effect on a health of the patient.

In some examples, a ratio of a ventilation volume to an air pressure is determined based on a current breath. In various examples, a ratio of a ventilation volume to an air pressure is determined based on individual ones of at least one previous breath. The volume to pressure ratio between consecutive breaths becoming greater, for example, may indicate the volume is increasing, the pressure is decreasing, or a combination thereof. The volume increasing, the pressure decreasing, or the combination thereof, may indicate, for example, the leak is slowing down and/or remedied. The volume to pressure ratio between consecutive breaths becoming lesser, for example, may indicate the volume is decreasing, the pressure is increasing, or a combination thereof. The volume decreasing, the pressure increasing, or the combination thereof, may indicate, for example, the leak is slowing down and/or remedied.

The volume to pressure ratio of the current breath, for example, is compared to the volume to pressure ratio(s) of the previous breath(s) to determine if a seal of the medical device 100 to the face of the patient is trending worse or better. The seal trending better may indicate, for example, that the user is addressing the leak and/or whether the cause of the leak is becoming less problematic. The seal trending worse may indicate a cause of the leak is still present, for example, The seal trending worse may indicate, for example, whether the cause of the leak potentially becoming more problematic.

In some examples, the medical device 100 may determine a lung compliance of the patient. Lung compliance, for instance, is a change in volume of air in the lungs with respect to changes in airway pressure. In some implementations, the medical device 100 includes a flow sensor configured to detect a flow of air into the airway and/or out of the airway. For example, an in-line bidirectional flow sensor may be physically coupled to the airway adaptor and/or a tube in a fluidic circuit within the medical device 100. The medical device 100 may be configured to calculate a volume of air into or out of the lung(s) of the patient by integrating the flow measurements with respect to time. In some examples, the medical device 100 further includes a pressure sensor configured to detect a pressure of air in the lung(s) of the patient by detecting a pressure within the fluidic circuit of the medical device 100. In various implementations, the medical device 100 estimates lung compliance of the patient by dividing the change in volume by the change in pressure. In various cases, the medical device 100 is configured to output an indication of the lung compliance, or to analyze a condition of the patient based on the lung compliance.

The alert warning the user to check for the leak is output, for example, based on the seal not trending better or trending worse, for example, over a number of ventilations (e.g., 2 ventilations, 4 ventilations, etc.). For example, the alert is not output, and/or the alert ends, for cases in which it was previously being output, based on the seal trending better, for example, over a number of ventilations (e.g., 2 ventilations, 4 ventilations, etc.). In some instances, the number of ventilations being utilized to output or not output the alert, for example, is a relatively lower number of ventilations if a rate of the trending of the leak is greater than a threshold rate, or a relatively greater number of ventilations if a rate of the trending of the leak is lower than a threshold rate.

In some instances, the medical device 100 being used in an ambulance traveling over uneven terrain or a windy road, or the medical device 100 being used in the field, such as an environment that is noisy, chaotic, etc., where the user or the medical device 100 may be bumped, knocked around, impacted by an object, etc., may result in the medical device 100 becoming askew, or may result in a portion of the mask of the medical device 100 becoming separated from the face of the patient. At least one metric that includes the mask seal rating algorithm result, the ratio of the ventilation volume to the air pressure, the inspired volume versus expired volume difference, the capnography, etc., for example, may be used to enable the user to accurately determine whether the leak is present even in poor conditions during use in the ambulance or in the field.

In various cases, the medical device 100 includes, and/or is connected via a wireless or wired connection, to one or more other sensors. Examples of the other sensors include, for instance, at least one pressure sensor within the cover (e.g., the air bladder) of the mask, at least one capacitive sensor in, or on a surface of, the mask, at least one resistive sensor in, or on a surface of, the mask, at least one pressure sensor in, or on a surface of, the mask, a chest compression altitude sensor, and so on. The other sensor(s), for example, are on a rim of the mask. The rim, for instance, which may include an outermost portion of the mask, may include a material that is the same as, or different from, at least one other material of the mask. In such an instance or another instance, the material of the rim may be of the same type as, or a different type from, at least one other type of material of the mask. In various cases, the material of the rim may be softer than, more pliable than, more firm than, more rigid than, etc., the material of any other portion of the mask. In various cases, the rim of the mask is flexible and configured to achieve a fluid-tight seal on the face of the patient. The other sensor(s), for example, detected at least one electrical parameter. The capacitive sensor(s), for example, detect the electrical parameter(s) that includes at least one capacitance (e.g., electrical capacitance level being greater than a threshold capacitance level) or at least one change in capacitance based on an object in contact with the capacitive sensor(s). The resistive sensor(s), for example, detect the electrical parameter(s) that includes at least one resistance (e.g., electrical resistance level below a threshold resistance level) or at least one change in resistance based on an object in contact with the resistive sensor. Any of the parameter(s) is detected at inspiration or expiration of the air.

In some cases, the medical device 100 and the monitoring device are connected to an intermediary device 106. The medical device 100 exchanges communications with the monitoring device 102 via a wireless connection 108 and/or a wired connection 110. The medical device 100 and/or the monitoring device exchange communications with the intermediary device 106 via a wireless connection and/or a wired connection. The wireless connection and/or the wired connection by which the medical device 100 and/or the monitoring device exchange communications with the intermediary device 106, for examples, are implemented in a similar way as the wireless connection 108 and/or the wired connection 110. For example, the wired connection by which the medical device 100 and/or the monitoring device exchange communications with the intermediary device 106 includes the wired connection 110, and/or the wireless connection by which the medical device 100 and/or the monitoring device exchange communications with the intermediary device 106 includes the wireless connection 108.

In various cases, the medical device 100 includes a user interface (UI) that outputs the physiological parameter(s). As used herein, the terms “user interface,” “UI,” and their equivalents, can refer to at least one hardware element that is configured to interact with a user by outputting signals to the user and/or receiving signals from the user. The UI of the medical device 100 outputs the physiological parameter(s) visibly and/or audibly. For instance, the UI of the medical device 100 includes a display, an indicator, a speaker, a haptic feedback device, or a combination thereof that conveys the physiological parameter(s). In some examples, the display of the medical device 100 outputs an air flow waveform representing the air flow data flow over time, and an air pressure waveform representing the air pressure data pressure over time.

In various examples, the medical device 100 presents, by the display, a numeric value, and/or a visual representation of any type, of any of the physiological parameter(s), in a similar way as for the waveform. In those or other examples, the medical device 100 audibly outputs, by the UI, a numeric value, and/or a representation of any type, of any of the physiological parameter(s), in a similar way as for the waveform. In various cases, the medical device 100 outputs, visibly and/or audibly, and/or in any way, any number of the physiological parameter(s), or a combination thereof. In various cases, the medical device 100 outputs, visibly, audibly, or otherwise, any numeric value and/or representation of any value computed utilizing any number of the physiological parameter(s) discussed herein.

In various cases, the monitoring device 102 includes a UI (e.g., a display, an indicator, a speaker, a haptic feedback device, or a combination thereof) that outputs the physiological parameter(s). The UI of the monitoring device 102 outputs the physiological parameter(s) visibly and/or audibly. For instance, the UI of the monitoring device 102 includes a display, a speaker, or a haptic feedback device that conveys the physiological parameter(s). In some examples, the display of the monitoring device 102 outputs the air flow waveform representing the air flow data flow over time, and the air pressure waveform representing the air pressure data pressure over time. The UI of the monitoring device 102 is controlled to duplicate, and/or be operated in the same way as, the medical device 100. For instance, the air flow waveform and/or the air pressure waveform output by the monitoring device 102 are the same as the air flow waveform and/or the air pressure waveform output by the medical device 100.

To exchange data, the medical device 100, the monitoring device 102, and/or the intermediary device 106 are configured to establish and/or communicate via at least one communication channel. As used herein, the term “communication channel,” and its equivalents, may refer to a medium over which a first endpoint (e.g., a sender) transmits information to one or more second endpoints (e.g., receivers). Examples of communication channels include wired connections (also referred to as “wired interfaces”), such as Ethernet, USB, HDMI, or fiber optic paths via physical wires and/or cables, as well as wireless connections (also referred to as “wireless interfaces”), such as Institute of Electronics and Electrical Engineers (IEEE) (e.g., WI-FI, BLUETOOTH, etc.) or 3^{rd} Generation Partnership Program (3GPP) (e.g., Long Term Evolution (LTE), New Radio (NR), etc.) connections. As used herein, the term “endpoint,” and its equivalents, may refer to an entity that is configured to transmit and/or receive data. Examples of endpoints include user equipment (UE) (e.g., mobile phones, tablet computers, etc.), computers, base stations, access points (Aps), servers, compute nodes, medical devices, Internet of Things (IoT) devices, and the like.

In some implementations, the communication channel(s) between the medical device 100, the monitoring device 102, and/or the intermediary device 106 are established when the medical device 100, the monitoring device 102, and/or the intermediary device 106 are paired. As used herein, the term “paired,” and its equivalents, may refer to a state of multiple devices that have a shared link key that enables each device to cryptographically authenticate data it receives from any other device among the multiple devices.

In particular cases, data transmitted between the medical device 100, the monitoring device 102, and/or the intermediary device 106 may include encrypted data. The data may be encrypted (e.g., translated from an unencoded format into another format (e.g., an encoded format)) and transmitted by any of the medical device 100, the monitoring device 102, and/or the intermediary device 106. The encrypted data be received and decrypted (e.g., translated from an encoded format into another format (e.g., an unencoded format)) by any of the medical device 100, the monitoring device 102, and/or the intermediary device 106.

Although the medical device 100, the monitoring device 102, and/or the intermediary device 106 are configured to establish communications and/or to communicate, as discussed above in the current disclosure, it is not limited as such. In various cases, any of the medical device 100, the monitoring device 102, the intermediary device 106, any other device (e.g., an oximetry sensor), or a combination thereof, communicates (e.g., transmits and/or receives), directly or indirectly (e.g., exchanges directly and/or routes), any data of any type with any of the medical device 100, the monitoring device 102, the intermediary device 106, any other device, or a combination thereof.

Although various types of data may be presented by the UI of the medical device 100, and/or transmitted by the transceiver, as discussed above in the current disclosure, it is not limited as such. In some cases, similar data to any of any of the data being presented by the UI and/or output in any way may be transmitted by the transceiver, and/or utilized to manage the transmitter to transmit data in a similar way to how the UI is utilized, for purposes of implementing the medical device 100 for performing any of the techniques discussed herein. In some cases, similar data to any of any of the data being transmitted by the transceiver and/or output in any way may be presented by the UI, and/or utilized to manage the UI to present data in a similar way to how the transceiver is utilized.

Although various types of input data (e.g., user input data) may be received by the UI of the medical device 100, and/or received by the transceiver (e.g., received as user input via a communication from a remote device being operated by a user), as discussed above in the current disclosure, it is not limited as such. In some cases, similar input data to any of the input data being received by the UI and/or received in any way may be received by the transceiver, and/or utilized to manage the transmitter for receiving the input data in a similar way to how the UI is utilized, for purposes of implementing the medical device 100 for performing any of the techniques discussed herein. In some cases, similar input data to any of the input data being received by the transceiver and/or received in any way may be received by the UI, and/or utilized to manage the UI for receiving the input data in a similar way to how the transceiver is utilized.

Although the various threshold(s) may be utilized in various ways along with operation of the medical device 100, as discussed above in the current disclosure, implementations are not so limited. In some cases, the medical device 100 is configured to compare one or more values described herein (e.g., the value of one or more ventilation parameters, blood oxygenation, or other parameters) to bracketed thresholds. For example, the medical device 100 is configured to determine whether an example parameter is greater than a first threshold and/or whether the example parameter is less than a second threshold. If the medical device 100 determines that the parameter is greater than the first threshold or less than the second threshold, then the medical device 100 may conditionally output an alert or perform one or more additional functions described herein. In some implementations, the first threshold is greater than the second threshold. In some cases, the second threshold is greater than the first threshold.

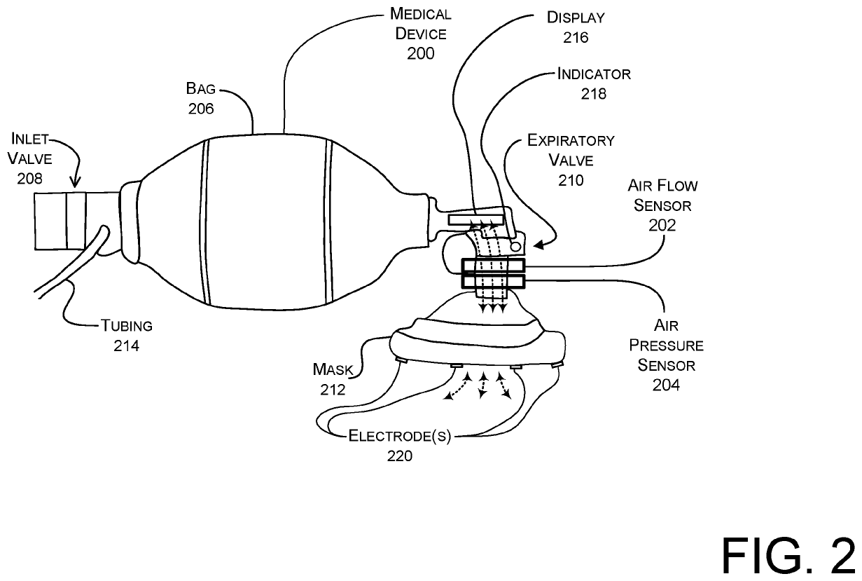

FIG. 2 illustrates an example illustration of a medical device 200 including an air flow sensor 202 and an air pressure sensor 204. In some examples, the medical device 200 is the medical device 100 described above with reference to FIG. 1.

In various implementations, the medical device 200 includes a processor. The processor, for example, performs any operations utilized to control and/or operate the medical device 200.

In various examples, the medical device 200 includes a bag 206, an inlet valve 208, an expiratory valve 210, a mask 212. The air flow sensor 202 and an air pressure sensor 204, for example, are in line with the bag 206 and the mask 212 of the medical device 200. In some cases, the mask 212 is configured to be in contact with a face of a patient. In some examples, the medical device 200 includes tubing 214, through which air flows during ventilation.

In some examples, the inlet valve 208 allows air from the environment to enter if fresh gas flow is inadequate. The expiratory valve 210, for example, forces air through into lungs of the patient in response to the mask 212 being applied to the face of the patient and the bag 206 being squeezed. In various cases, the expiratory valve 210 allows the lungs of the patient to deflate in response to the mask 212 being applied to the face of the patient and the bag 206 being allowed to inflate (e.g., self-inflate).

In various cases, the medical device 200 includes at least one other sensor. Examples of the other sensor(s), for instance, include a CO₂ sensor, an O₂ sensor, a humidity sensor, a temperature sensor, or the like. The other sensor(s), for example, are in line with the bag, the mask, and the valve of the medical device 200.

In some examples, the bag 206 is fluidly connected to the mask 212. The bag 206 and the mask 212 enclose a space that is fluidly connected to an airway of the patient. A ventilation (e.g., a first ventilation) occurs when the bag 206 is contracted and released a first time. In various instances, ventilation (e.g., a second ventilation) occurs when the bag 206 is contracted and released a second time.

In some cases, the medical device 200 detects at least one physiological parameter and/or determines at least one metric to determine whether a leak is present in the space of the medical device that is fluidly connected to the airway of the patient. Examples of the physiological parameter(s) include, for instance, a flow rate, an air pressure, a CO₂ level (e.g., at least one of a partial pressure of CO₂, a partial pressure of EtCO₂, and so on), an O₂ level (e.g., a partial pressure of O₂), a humidity, a temperature, or the like.

In various cases, the medical device 200 determines ventilation volumes by analyzing the flow of air during ventilations when the bag is contracted and released. As used herein, the term “ventilation,” and its equivalents, can refer to an operation of a ventilation device and a process based on the ventilation device operation including a ventilation cycle with a bag being sequentially contracted and released causing the air to flow into and out of lungs of a patient. The ventilation cycle, for example, includes expiration and inspiration of air based on the bag being contracted and released. Additionally or alternatively, as used herein, the term “ventilation,” and its equivalents, can refer to an operation of a ventilation device and a process based on the ventilation device operation including a bag being contracted causing air to flow into lungs of a patient. Additionally or alternatively, as used herein, the term “ventilation,” and its equivalents, can refer to an operation of a ventilation device and a process based on the ventilation device operation including a bag being released causing air to flow out of lungs of a patient.

In various examples, the medical device 200, for example, determines ventilation volume (e.g., a first ventilation volume) by analyzing the flow of air during the first ventilation, and a ventilation volume (e.g., a second ventilation volume) by analyzing the flow of air during the second ventilation. In some examples, the medical device 200 identifies a ratio (e.g., a first ratio) between the first ventilation volume and the pressure during the first ventilation, and another ratio (e.g., a second ratio) between the second ventilation volume and the pressure during the second ventilation. The medical device 200, for example, determines a discrepancy between the first ratio and the second ratio, and, in response to determining the discrepancy between the first ratio and the second ratio, determines that a leak is present between the mask 212 and the face of the patient.

In various cases, the medical device 200 includes a user interface (UI) (e.g., a display, an indicator, a speaker, a haptic feedback device, or a combination thereof) that is controlled based on the physiological parameter(s). For example, the UI includes a display 216 and an indicator 218.

In some examples, the medical device 200 causes the display 216 to output an alert indicating that the leak is present between the mask and the face of the patient. The alert, for example, is output temporarily for a predetermined time period, or, in some implementations, until the leak stops.

In some examples, the medical device 200 causes the display 216 to output physiological parameter data representing the physiological parameter(s). The physiological parameter data is output, for example, as at least one of text, an element of a graph, a geometric shape, and so on.

In some examples, the medical device 200 causes the indicator 218 to be activated (e.g., illuminated) indicating that the leak is present. The indicator 218, for example, is caused to be activated according to a threshold period (e.g., a first threshold period of time) as a solid illumination, a periodic illumination (e.g., blinking), or a patterned illumination (e.g., an illumination timed to a pattern representing words in a dialogue, or a pattern representing notes or beats in, or a rhythm of, a song), or to be activated as a color (e.g., a red color) representing the leak. The indicator 218, for example, is caused to be activated according to a threshold period of time (e.g., a second threshold period of time) as a solid illumination or a periodic illumination (e.g., blinking), or to be activated as a color (e.g., a green color) representing the leak ending (e.g., slowing down, having fully ended, etc.). An illumination type (e.g., a first illumination type) representing the leak, for example, is different from an illumination type (e.g., a second illumination type) representing the leak ending. In some cases, the illumination type of the illumination representing the leak includes the indicator 218 being activated until a period time meets or exceeds the first threshold period of time using the periodic illumination with the red color; and the illumination type of the illumination representing the leak ending includes the indicator 218 being activated until a period time meets or exceeds the second threshold period of time using the solid illumination with the green color. For example, the indicator 218 is activated for an indefinite period of time (e.g., a trigger-based period of time) until the leak ends, at which point the indicator 218 is activated for a predetermined period of time (e.g., 10 seconds, 30 seconds, 1 minute, etc.).

In some examples, the medical device 200 causes the speaker to be activated according to a threshold period of time (e.g., a first threshold period of time) to output audio (e.g., a horn, a ding, a bell, and so on) at an audio volume (e.g., a first audio volume) indicating that the leak is present. The medical device 200, for example, causes the speaker to be activated according to a threshold period of time (e.g., a second threshold period of time) to output audio (e.g., the horn, the ding, the bell, and so on) at an audio volume (e.g., a second audio volume) indicating the leak ending (e.g., slowing down, having fully ended, etc.). In some cases, the first period of time is different from the second period of time. In some cases, the first audio type is different from the second audio type. In some cases, the first audio volume is different from the second audio volume. The audio being output for at least one of the leak being present or the leak ending can be output steadily, periodically, or according to a pattern (e.g., a sound timed to a pattern representing words in a dialogue, or a pattern representing notes or beats in, or a rhythm of, a song). For example, the audio type for the leak includes a horn being output steadily for an indefinite period of time (e.g., a trigger-based period of time) until the leak ends, at which point the audio type includes a bell being output periodically for a predetermined period of time (e.g., 10 seconds, 30 seconds, 1 minute, etc.).

In some examples, the medical device 200 causes the speaker to output audio including dialogue. A dialogue type (e.g., a first dialogue type) output based on the leak being present, for example, is different from a dialogue type (e.g., a second dialogue type) output based on the leak ending (e.g., slowing down, having fully ended, etc.). In some cases, the dialogue type output based on the leak being present includes a notification or explanation to inform the user that the leak is present; and the dialogue type output based on the leak ending includes a notification or explanation to inform the user of the leak ending.

In some examples, the medical device 200 causes the haptic feedback device to provide haptic feedback (e.g., vibrate, move, or the like). The medical device 200, for example, causes the haptic feedback device to be activated according to a threshold period of time (e.g., a first threshold period of time) to move at a movement size (e.g., a first movement size) indicating that the leak is present. The medical device 200, for example, causes the haptic feedback device to be activated according to a threshold period of time (e.g., a second threshold period of time) to move at a movement size (e.g., a second movement size) indicating the leak ending (e.g., slowing down, having fully ended, etc.). The haptic feedback device for at least one of the leak being present or the leak ending can be controlled to move steadily, periodically, or according to a pattern (e.g., a movement timed to a pattern representing words in a dialogue, or a pattern representing notes or beats in, or a rhythm of, a song). For example, the movement type for the leak is at least one of a relatively faster or a relatively greater movement level for an indefinite period of time (e.g., a trigger-based period of time) until the leak ends, at which point the movement type includes a relatively slower or a relatively lesser movement level, periodically for a predetermined period of time (e.g., 10 seconds, 30 seconds, 1 minute, etc.).

In some examples, the UI includes the display 216 outputting the alert visually or audibly as an escalating alert having a first alert level at an initial time being lower than a second alert level at a subsequent time. In various examples, the UI identifies an ignore alert selection received via user input to the UI. The alert, for example, is silenced based on the ignore alert selection.

In various examples, the medical device 200 includes at least one sensor. Examples of the sensor(s) include at least one electrode 220. The electrode(s) 220, for example, are disposed on an edge or a rim of the mask and configured to detect an electrical signal. The medical device 200, for example, detects a separation between the edge of the mask and the face of the patient by analyzing the electrical signal. Examples of the sensor(s) included in the medical device 200, additionally or alternatively to the electrode(s) 220, include at least on sensor in an interior of the mask 212 to detect a pressure based on the mask being held against the face of the patient. For example, the sensor(s) in the interior of the mask 212 may be included in the cover, the cushion (e.g., the inflatable air cushion), the air bladder, the pillow, the mat, or the like. Additionally or alternatively, examples of the sensor(s) include at least on sensor between the mask 212 and a portion of the medical device 200 to detect a pressure based on the mask being held against the face of the patient. For example, the sensor(s) between the mask 212 and a portion of the medical device 200 may be between the mask and a neck of the medical device 200.

In some cases, the medical device 200 includes an accelerometer configured to detect a movement of a chest of the patient. The medical device 200, for example, detects the separation between the mask and the face of the patient by analyzing the movement of the chest of the patient in view of the flow or pressure. In various cases, the medical device 200, in response to detecting the separation between the edge of the mask and the face of the patient, changes at least one of a UI characteristic (e.g., a first UI characteristic) or an intensity (e.g., a first intensity) of an alert (e.g., a first alert) to be another UI characteristic (e.g., a second UI characteristic) or another intensity (e.g., a second intensity). For example, the medical device 200 increases the first intensity to the second intensity. The medical device 200, for example, causes the UI (e.g., the display, the indicator, the speaker, the haptic feedback device, or a combination thereof) to output an alert (e.g., a second alert) indicating that the mask is separated from the face of the patient.

In various cases, the medical device 200 determines that the leak has greater than a threshold severity by analyzing the physiological parameter(s). In response to determining that the leak has greater than the threshold severity, the medical device 200, for example, changes at least one of the UI characteristic(s) or the intensity of the first alert. The medical device 200, for example, causes the display to output a second alert indicating that the leak has greater than the threshold severity.

In various examples, changing the UI characteristic(s) based on the leak having greater than the threshold severity includes changing at least one of a color of the illumination of the indicator or the output of the display, a display format (e.g., the text, the element of the graph, the geometric shape, and so on) of the output of the display 216, a brightness level of the illumination of the indicator 218, a volume of the audio output by the speaker, a size of the text, the graph element, the geometric shape, and so on, output by the display 216, a brightness level of the indicator 218, a brightness level of the text, the graph element, the geometric shape, and so on, output by the display 216, a movement level of the haptic feedback device, a type (e.g., solid, patterned, etc.) of the audio output by the speaker, a type (e.g., solid, blinking, etc.) of the illumination of the indicator 218, a type (e.g., solid, patterned, etc.) of the movement of the haptic feedback device, or a type (e.g., solid, blinking, etc.) of the text, the graph element, the geometric shape, and so on, output by the display 216, any other UI characteristic, or any combination thereof. In some cases, a level of the UI characteristic(s) for the first alert is changed to a greater level for the second alert.

In various cases, based on determining at a time (e.g., a first time) that the leak has greater than the threshold severity, the medical device 200 determines at a time (e.g., a second time being subsequent to the first time) that the leak has less than the threshold severity by analyzing the physiological parameter(s). In response to determining that the leak has less than the threshold severity, the medical device 200, for example, changes at least one of the UI characteristic(s) or the intensity of the second alert. The medical device 200, for example, causes the display to output a third alert (e.g., the intensity of the second alert is changed to a third intensity of the third alert). In some cases, a level of the UI characteristic(s) for the second alert is changed to a greater level for the third alert. The UI characteristic(s) for the third alert, for example, can be controlled to be at a same level as the UI characteristic(s) for the first alert.

In some examples, the medical device 200 determines a percentage of air that is leaking based on the physiological parameter(s). The medical device 200, for example, performs a comparison to compare a ventilation volume to a predetermined expected ventilation volume, an air flow to a predetermined expected air flow, an air pressure to a predetermined air pressure. The medical device 200 determines a metric that includes an air flow percentage based on the comparison, based on a table identifying the air flow percentage as corresponding to at least one of the ventilation volume, the air flow, or the air pressure.

In various cases, additional notifications, which are different from the notification of the leak ending or having ended, are output to inform the user of the leak slowing down. The additional notifications include, for example, an intermediary notification informing the user of the leak slowing down and the percentage of air that is leaking.

In some implementations, the medical device 200 identifies sensor status data associated with the air flow sensor 202 and/or the air pressure sensor 204. The sensor status data, for example, includes a sensor identifier, a sensor name, an identification number (or “ID number”), a sensor type, sensor calibration information, sensor compatibility information, a sensor lifespan, a sensor version number, and so on, or a combination thereof.

In various examples, the sensor status data is stored in the medical device 300. Additionally or alternatively, the sensor status data is transmitted to the monitoring device 102 and/or the intermediary device 106. In some examples, at least some data included in the sensor status data is presented by the display 316, the display in the monitoring device 102, and/or the display in the intermediary device 106.

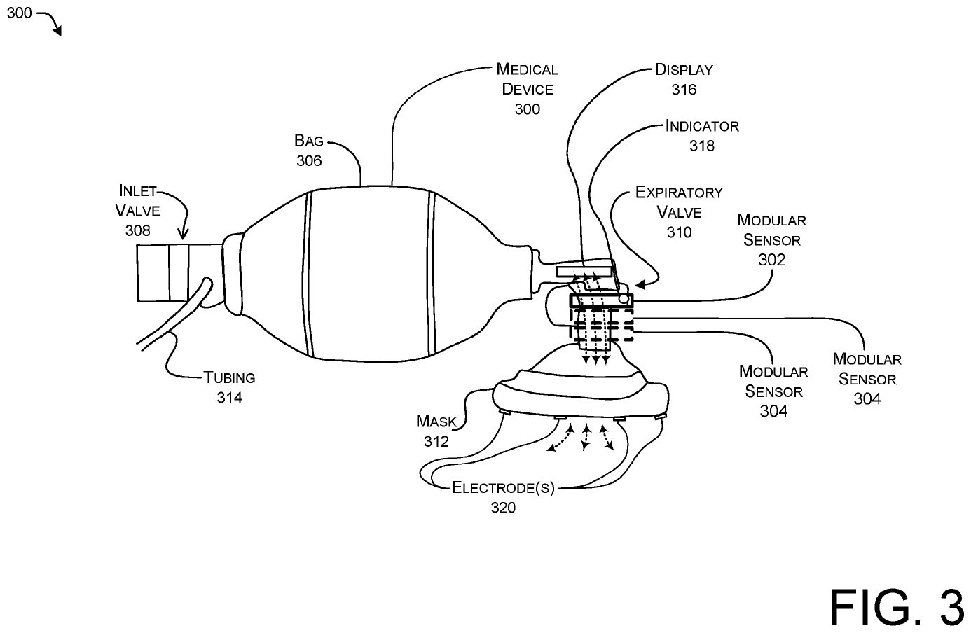

FIG. 3 illustrates an example illustration of a medical device including one or more modular sensors. In some examples, the medical device 300 is the medical device 100 described above with reference to FIG. 1. In some implementations, the medical device 300 is the medical device 200 described above with reference to FIG. 2, but without at least one of the air flow sensor 202 or the air pressure sensor 204.

In various implementations, the medical device 300 includes a processor. The processor, for example, performs any operations utilized to control and/or operate the medical device 300.

In various examples, the medical device 300 includes a bag 306, an inlet valve 308, an expiratory valve 310, a mask 312, a tubing 314, a display 316, and an indicator 318. In some implementations, the bag 306, the inlet valve 308, the expiratory valve 310, the mask 312, the tubing 314, the display 316, and the indicator 318 are the bag 206, the inlet valve 208, the expiratory valve 210, the mask 212, the tubing 214, the display 216, and the indicator 218 described above with reference to FIG. 2.

In various cases, the medical device 300 includes at least one connector to which at least one modular sensor is coupled. For example, the modular sensor(s) include a modular sensor 302 currently coupled to the medical device 300, and at least one modular sensor 304 to be coupled to the medical device 300. In some examples, at least one of the modular sensor 302 or the modular sensor(s) 304 can be implemented in a similar way as the air flow sensor 202, the air pressure sensor 204, or any of the other sensor(s), such as the CO₂ sensor, the O₂ sensor, the humidity sensor, the temperature sensor, or the like, except by being modular instead of fixed. In some examples, at least one of the modular sensor(s) 304 can be implemented in a similar way as the modular sensor 302.

In some instances, the modular sensor 302 is detachably coupled to the medical device 300. For example, the modular sensor 302 being detachably coupled is connected removably, temporarily, and so on, to the medical device 300. In some implementations, the modular sensor 302 is mechanically and/or electrically coupled to the medical device 300.

The connector, for example, includes at least one thread, at least one rivet, at least one clasp, at least one tong, at least one fastener, at least one hook, at least one buckle, at least one pin, at least one screw, or the like, or any combination thereof. The modular sensor 302, for example, includes at least one thread, at least one rivet, at least one clasp, at least one tong, at least one fastener, at least one hook, at least one buckle, at least one pin, at least one screw, or the like, or any combination thereof. In various implementations, the thread(s), the rivet(s), clasp(s), the tong(s), the fastener(s), the hook(s), the buckle(s), the pin(s), the screw(s), or the like, included in the connector(s) and the modular sensor 302 are utilized to couple the modular sensor 302 to the connector, and thereby, to the medical device 300.

In some instances, the modular sensor 302 is coupled to the medical device 300 without utilizing the connector(s), for example, in such cases in which the medical device 300 does not include any modular sensor connectors. In various examples, the modular sensor 302 is coupled to at least one portion of the medical device 300, the portion(s) including a tube, a sleeve, a cover, a shell, the mask 312, the bag 306, any other portion of the medical device 300, or any combination thereof. The modular sensor 302, for example, is coupled to the medical device 300 based on the thread(s), the rivet(s), clasp(s), the tong(s), the fastener(s), the hook(s), the buckle(s), the pin(s), the screw(s), or the like, being coupled to the portion(s) of the medical device 300.

In various implementations, the modular sensor 302, for example with instances in which the modular sensor 302 does not include the thread(s), the rivet(s), clasp(s), the tong(s), the fastener(s), the hook(s), the buckle(s), the pin(s), the screw(s), or the like, is shaped to be form fitted around the portion(s) of the medical device 300. For example, the modular sensor 302 has a material that is pliable, bendable, elastic, adherent, and so on, for coupling the modular sensor 302 to the medical device 300. Additionally or alternatively, the modular sensor 302, for example, includes at least one connector that is pliable, bendable, elastic, adherent, and so on, for coupling the modular sensor 302 to the medical device 300. For instance, with examples in which at least one of the modular sensor 302, or the connector of the modular sensor 302, is adherent, an adhesive covers, or is applied to, at least a portion of the modular sensor 302 or at least a portion of the connector.

In some examples, the medical device 300 includes at least one of a slot, a compartment, a hole, an inlet, a receptacle, and so on, used to couple the modular sensor 302 to the medical device 300. The modular sensor 302, for example, is firmly or loosely placed in the slot, the compartment, the hole, the inlet, the receptacle, and so on, or a combination thereof.

In some examples, the modular sensor 302 is coupled to at least one other modular sensor (e.g., the modular sensor(s) 304). For example, the modular sensor 302 is coupled via the thread(s), the rivet(s), clasp(s), the tong(s), the fastener(s), the hook(s), the buckle(s), the pin(s), the screw(s), or the like, or a combination thereof, to the thread(s), the rivet(s), clasp(s), the tong(s), the fastener(s), the hook(s), the buckle(s), the pin(s), the screw(s), or the like, or a combination thereof, of the other modular sensor(s). In some cases, the modular sensor 302 is coupled to the other modular sensor(s), but not coupled directly to the medical device 300. In various cases, the modular sensor 302 is coupled to the other modular sensor(s), and to the medical device 300.

In some examples, the modular sensor 302 and the other modular sensor(s) being coupled together form a modular sensor assembly. The modular sensor assembly, for example, is detachably coupled to the medical device 300.

In various implementations, the modular sensor 302 is coupled to, inserted in, or integrated with, a case, a cover, a bracket, a shell, or the like, or a combination thereof. The case, the cover, the bracket, the shell, or the like, or the combination thereof, for example, of the modular sensor 302 is detachably coupled to the medical device 300. In some examples, the modular sensor assembly is coupled to, inserted in, or integrated with, a case, a cover, a bracket, a shell, or the like, or a combination thereof. The case, the cover, the bracket, the shell, or the like, or the combination thereof, for example, of the modular sensor assembly is detachably coupled to the medical device 300. In some instances, the modular sensor 302 and/or the modular sensor assembly is detachably or firmly coupled to at least one fixed sensor (e.g., the sensor(s) 104) and/or the medical device 200.

In some examples, the modular sensor 302 is electrically coupled to the medical device 300 by direct or indirect contact. The electrical coupling, for example, is wired or wireless. In various examples, direct contact includes an electrical connector of the modular sensor 302 being physically coupled with an electrical connector of the medical device 300. In some implementations, the modular sensor 302 includes a transceiver.

In some implementations, the modular sensor 302 and the medical device exchange communications based on the electrical connector(s) of the modular sensor 302 being conductive, capacitive, inductive, or a combination thereof. In some implementations, for example with instances in which the modular sensor 302 does not include an electrical connector being physically coupled to an electrical connector of the medical device 300, communications between the modular sensor 302 and the medical device 300, for example, are induced and/or exchanged magnetically, capacitively, inductively, or a combination thereof. The physiological parameter(s), for example, detected by the modular sensor 302 are identified by, and/or transmitted to, the medical device 300.

In some examples, the modular sensor 302 is electrical coupled to at least one of the monitoring device 102 or the intermediary device 106 by at least one of a wired or wireless connection. In some examples, the wired and/or the wireless connection by which the modular sensor 302 exchanges communications with the monitoring device 102 and/or the intermediary device 106 is implemented in a similar way as the wired connection 110 and/or the wireless connection 108. For example, the wired connection by which the medical device 100 and/or the monitoring device exchange communications with the modular sensor 302 includes the wired connection 110, and/or the wireless connection by which the medical device 100 and/or the monitoring device exchange communications with the modular sensor 302 includes the wireless connection 108.

In some implementations, the medical device 300 identifies the modular sensor 302 being coupled to the medical device 300. The medical device 300, for example, identifies modular sensor status data associated with the modular sensor 302 based on the modular sensor 302 being coupled to the medical device 300. The modular sensor status data, for example, includes a sensor identifier, a sensor name, an identification number (or “ID number”), a sensor type, sensor calibration information, sensor compatibility information, a sensor lifespan, a sensor version number, and so on, or a combination thereof. In various examples, the modular sensor status data includes a flag set based on the modular sensor 302 being coupled to the medical device 300.

In various cases, the medical device 300 identifies the modular sensor assembly being coupled to the medical device 300. The medical device 300, for example, identifies modular sensor status data associated with at least one modular sensor, such as the modular sensor 302, in the modular sensor assembly.

In various examples, the modular sensor status data is stored in the medical device 300. Additionally or alternatively, the modular sensor status data is transmitted to the monitoring device 102 and/or the intermediary device 106. In some examples, at least some status data included in the modular sensor status data is presented by the display 316, the display in the monitoring device 102, and/or the display in the intermediary device 106. The physiological parameter(s), for example, detected by the modular sensor 302 are transmitted to the monitoring device 102 and/or the intermediary device 106.

In various examples, the modular sensor 302 and/or the modular sensor assembly is activated by the medical device 300, the monitoring device 102, and/or the intermediary device 106. In some examples, the modular sensor 302 and/or the modular sensor assembly is deactivated by the medical device 300, the monitoring device 102, and/or the intermediary device 106. The activation and/or the deactivation, for example, is performed to conserve power in a power supply device of the medical device 300. The activation and/or the deactivation, for example, is automatically controlled by the medical device 300, the monitoring device 102, and/or the intermediary device 106. The activation and/or the deactivation, for example, is automatically controlled, for example, based on coupling and/or decoupling of the modular sensor 302 and/or the modular sensor assembly to the medical device 300. The medical device 300, for example, transmits a signal (e.g., a status signal) to the monitoring device 102, activates an indicator of the medical device 300, and/or outputs status information via the display in the medical device 300, based on the modular sensor 302 being coupled to, or decoupled from, the medical device 300. The signal indicates, for example, the modular sensor 302 being coupled to, or decoupled from, the medical device 300.

In some implementations, the activation and/or the deactivation, for example, of the modular sensor is manually controlled by the medical device 300, the monitoring device 102, and/or the intermediary device 106 based on an activation or a deactivation selection received via user input to the medical device 300, the monitoring device 102, and/or the intermediary device 106. The activation or the deactivation selection is received, for example, via user input to at least one UI of the medical device 300, the monitoring device 102, and/or the intermediary device 106. The UI(s) of the medical device 300, the monitoring device 102, and/or the intermediary device 106 includes, for example, the display being a touchscreen. In some examples, the UI(s) of the medical device 300, the monitoring device 102, and/or the intermediary device 106 includes at least one keypad, at least one mouse, at least one scroll wheel, at least one button, at least one toggle stick, at least one joystick, at least one touchpad, and so on, or any combination thereof.

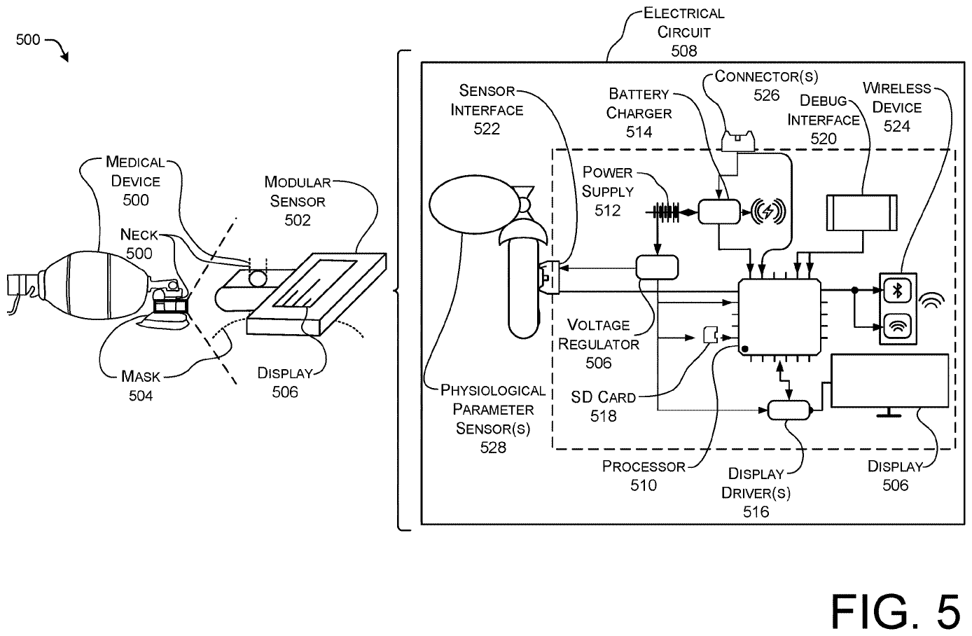

In some examples, the modular sensor 302 includes a display, a storage device (e.g., a battery), and a circuit. In some implementations the display in the modular sensor 302 is the display 506 described below with reference to FIG. 5.

In various examples, the circuit is included on a PCB. The PCB includes, for example, at least one of a processor, the storage device, a voltage regular, a transceiver, and so on, or any combination thereof, being connected to, and or integrated within, the circuit. In some examples, the PCB includes a bus and/or a microcontroller in addition, or alternatively to, the circuit and the processor. The battery charger, for example, includes a wireless battery charger including an inductive coil for wirelessly receiving power, an inductive coil for wirelessly transmitting power, or a combination thereof. In some examples, the PCB includes a charger connector and a data connector.

In various implementations, the PCB is coupled to a physiological parameter sensor by a connector. In some examples, the PCB is optically coupled to a physiological parameter sensor. A type of the physiological parameter sensor, for example, corresponds to a sensor type of the modular sensor 302. A type of physiological parameter, for example, detected by the physiological parameter sensor corresponds to the sensor type.

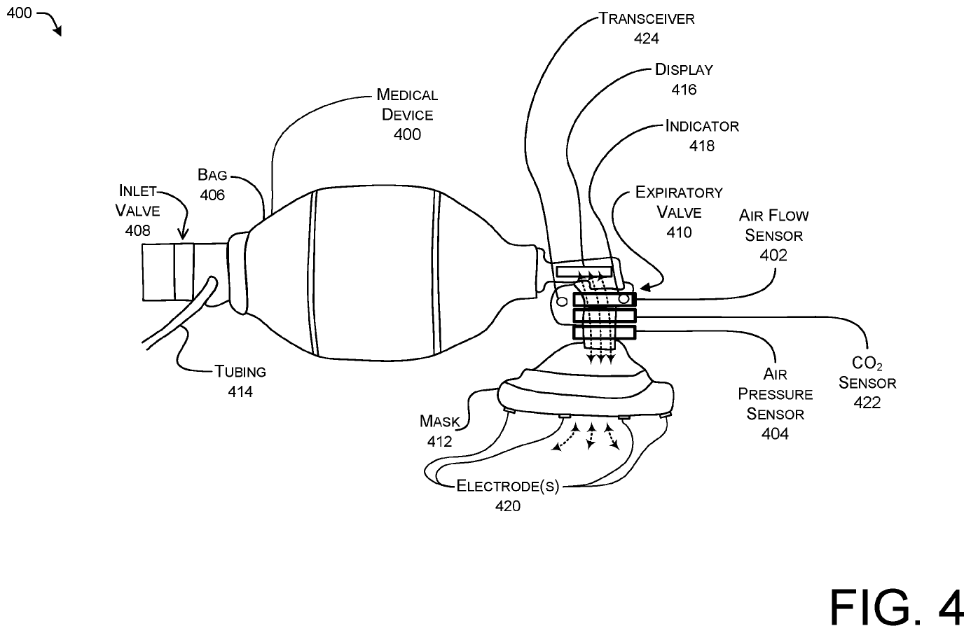

FIG. 4 illustrates an example illustration of a medical device including an air flow sensor, CO2 sensor, and an air pressure sensor. In some examples, the medical device 400 is the medical device 100, the medical device 200, or the medical device 300 described above with reference to FIGS. 1-3.

In various implementations, the medical device 400 includes a processor. The processor, for example, performs any operations utilized to control and/or operate the medical device 400.

In various examples, the medical device 400 includes a bag 406, an inlet valve 408, an expiratory valve 410, a mask 412, a tubing 414, a display 416, and an indicator 418. In some implementations, the bag 406, the inlet valve 408, the expiratory valve 410, the mask 412, the tubing 414, the display 416, the indicator 418, and the electrode(s) 420 are the bag 306, the inlet valve 308, the expiratory valve 310, the mask 312, the tubing 314, the display 316, the indicator 318, and the electrode(s) 320 described above with reference to FIG. 3. In some implementations, the bag 406, the inlet valve 408, the expiratory valve 410, the mask 412, the tubing 414, the display 416, the indicator 418, and the electrode(s) 420 are the bag 206, the inlet valve 208, the expiratory valve 210, the mask 212, the tubing 214, the display 216, the indicator 218, and the electrode(s) 220 described above with reference to FIG. 2.

In various examples, the medical device 400 includes an air flow sensor 402 and an air pressure sensor 404. In some implementations, the air flow sensor 402 and the air pressure sensor 404 are the air flow sensor 202 and the air pressure sensor 204 described above with reference to FIG. 2. In some various examples, the medical device 400 includes a CO₂ sensor 422.

In some examples, the air flow sensor 402 is coupled between the mask 412 and the bag 406. The air flow sensor 402, for example, detects a flow rate of air flowing between the mask 412 and the bag 406. In some examples, the air pressure sensor 404 is coupled between the mask 412 and the bag 406. The air pressure sensor 404, for example, detects a pressure of the air. In some examples, the CO₂ sensor 422 is coupled between the mask 412 and the bag 406. The CO₂ sensor 422, for example, detects a CO₂ level (e.g., at least one of a partial pressure of CO₂, a partial pressure of EtCO₂, and so on) in the air in a space of the medical device 400. The space of the medical device 400, for example, includes a space that is fluidly connected to the airway of the patient.

In some cases, the medical device 400 includes at least one sensor, such as an O₂ sensor, a humidity sensor, a temperature sensor, or the like. In some implementations, the sensor(s) are the sensor(s) 104. In various implementations, the sensor(s) detect the physiological parameter(s), including an O₂ level (e.g., a partial pressure of O₂), a humidity, a temperature, or the like.

In various implementations, the air flow sensor 402, the air pressure sensor 404, and the CO₂ sensor 422 are coupled in any order with respect to one another. In some examples, the air flow sensor 402, the air pressure sensor 404, and/or the CO₂ sensor 422 are integrated into a single sensor.

In some examples, the medical device 400 determines, based on the flow rate, the CO2 level, and the pressure, that a leak is present between the face and the mask. The processor, in response determining that the leak is present between the face and the mask, for example, causes the transceiver 424 to transmit a signal indicating the leak, and/or causes the display 416 to output an alert indicating the leak.

In some implementations, the medical device 400 determines whether the leak is present based on a metric that includes an end tidal ventilation volume, an average ventilation volume, or a combination thereof. The end tidal volume, an average volume, or a combination thereof, for example, is identified based on the flow rate. In various examples, the medical device 400 identifies the end tidal volume, the average volume, or the combination thereof, based on the flow rate being identified and integrated over time. The flow rate is integrated over time, for example, to calculate a ventilation volume. A ventilation volume, for example, including a current volume of air (in milliliters (mLs) being administered to a patient by the medical device 400 is identified. A ventilation volume, for example, including a current volume of air (e.g., mLs of air) being inhaled by a patient is identified. A ventilation volume, for example, including a current volume of air (in mLs) being exhaled by a patient is identified. In some examples, an end tidal volume is identified (e.g., captured) based on an end of a ventilation or a breath being taken by the patient (e.g., the end of the ventilation or the breath based on the air having been exhaled). In some cases, the end tidal volume is identified based on the end of the ventilation or the breath. The end tidal volume, for example, is identified at a ventilation cycle that includes the ventilation being taken, based on the ventilation volume(s).

In some examples, at least one metric that includes the end tidal volume is utilized to identify the average volume. The end tidal volume, for example, identified at the ventilation cycle is latched until a next ventilation cycle. In various cases, any number of the end tidal volume(s) are identified and latched. An average volume, for example, is identified based on the end tidal volume(s) identified at the ventilation cycle(s).

In various cases, whether the leak is present between the face and the mask is identified based on the end tidal volume, the average volume, or the combination thereof. The leak being present between the face and the mask is identified, for example, based on the average volume being less than a threshold average volume.

In some implementations, the medical device 400 determines whether the leak in the space that is fluidly connected to the airway of the patient is present based on a metric that includes a volume-flow graph. In various examples, the volume-flow graph is identified based on at least one ventilation volume. The ventilation volume(s), for example, include a volume based on an inspiratory cycle including an inspiration of the patient (e.g., air flowing in a first direction through the space, such as from the medical device 400 into the airway of the patient), a volume based on an expiratory cycle including an expiration of the patient (e.g., air flowing in a second direction through the space, such as from the airway of the patient into the medical device 400, wherein the second direction is different than the first direction), or any combination thereof. The volume-flow graph is identified, for example, based on a comparison between the volume for the inspiratory cycle and the volume for the expiratory cycle.

In various instances, the leak being present is identified based on the volume-flow graph, which includes the volume identified based on the inspiratory cycle and the volume based on the expiratory cycle. In some examples, the leak being present is identified based on a difference between the volume identified based on the inspiratory cycle and the volume based on the expiratory cycle being less than a threshold difference. In various examples, the leak being present is identified based on a volume (e.g., a first volume) identified based on an inspiratory cycle, another volume (e.g., a second volume) identified based on another inspiratory cycle, and a difference between the second volume and the first volume being greater than a threshold difference. In various examples, the leak being present is identified based on a volume (e.g., a third volume) identified based on an expiratory cycle, another volume (e.g., a fourth volume) identified based on another expiratory cycle, and a difference between the fourth volume and the third volume being greater than a threshold difference.

In various instances, the leak being present is identified based on the volume-flow graph that includes a local real-time volume-flow graph. The local real-time volume-flow graph, for example, is identified by the medical device 400 and presented by the display 416 dynamically (e.g., in real-time) in a position near the airway of the patient. The term “local,” with respect to the meaning of the local real-time volume-flow graph refers to, for example, the volume-flow graph being presented by the medical device 400, such as with the volume-flow graph being presented by the display 416. The local real-time volume-flow graph being presented by the display 416 in real-time includes, for example, the local real-time volume-flow graph being presented during operation of the medical device 400. The display 416, for example, has a resolution being greater than or equal to a threshold resolution, to present the volume-flow graph to be easily viewed by caregivers.

In various instances, the leak being present is identified based on a metric that includes a patient breath detection and respiration rate. The patient breath detection and respiration rate, for example, is identified based on the CO2 level (e.g., at least one of a partial pressure of CO₂, a partial pressure of EtCO₂, and so on). In some examples, a patient breath detection and respiration rate is identified based on a start of an inspiratory. In some examples, a patient breath detection and respiration rate is identified based on an end of an inspiratory. The patient breath detection and respiration rate(s), for example, are identified based on flow data including a detected flow rate at the start of the inspiratory, a detected flow rate at the end of the inspiratory, and so on. The patient breath detection and respiration rate(s) include, for example, a patient breath detection and respiration rate identified based on a start and an end of an expiration. The flow data, for examples, includes a detected flow rate at a start of an expiratory, a detected flow rate at an end of an inspiratory, and so on.

In some cases, the flow data, for example, is used to measure a duration of breath. The duration of the breath, for example, includes a duration of a inspiratory cycle between the start and the end of the inspiratory, a duration between the end of the inspiratory and the start of the expiratory, a duration of a expiratory cycle between the start and the end of the expiratory, a duration between the end of the expiratory and the start of the inspiratory, or any combination thereof. A respiration rate is calculated, for example, based on a reciprocal of an average duration of a number of most recent inspiratory and expiratory cycles (e.g., 8 most recent inspiratory and expiratory cycles). The leak being present is identified, for example, based on the respiration rate being less than a threshold respiration rate (e.g., a first threshold respiration rate) or greater than another threshold respiration rate (e.g., a second threshold respiration rate).

In various instances, the leak being present is identified based on a metric that includes a compression feedback through ventilation metric. The compression feedback through ventilation metric is identified, for example, based on the O2 level. The compression feedback through ventilation metric is identified, for example, based on noise in a capnogram. The capnogram is identified based on the detected CO2 level, for example, and includes at least one segment and at least one angle. The segment(s), for example, include an inspiratory segment and an expiratory segment. The angle(s), for example, including an alpha angle and a beta angle. A compression level in a predetermined amount of time, for example, are identified based on the noise in a capnogram. Compression feedback is identified using only a ventilation parameter based on the identified compression level. The leak being present is identified based on the compression feedback that includes the compression level being less than a threshold compression level, for example.

In various instances, the leak being present is identified based on a metric that includes an adaptive inspiratory-expiratory ratio. The adaptive inspiratory-expiratory ratio is identified, for example, based on oxygenation feedback. The oxygenation feedback, for example, is identified and includes an oxygenation feedback parameter that gives an impression of how effective respiration is performed. The oxygenation feedback includes, for example, an inspirated O₂ and an expired CO₂. A concentration is determined, for example, based the inspirated O₂ and the expired CO₂. In some examples, the leak being present is identified based on a difference between an inspirated O₂ concentration that includes the inspirated O₂ and an expired CO₂ concentration that includes the expired CO₂ being greater than a threshold difference. The inspirated O₂ concentration being too high with relation to the expired CO₂ concentration is, for example, indicative that the patient may have an airway issue based on the leak. The patient may have an airway issue may indicate, for example, that alveoli diffusion is not occurring properly.

In various instances, the leak being present is identified based on a metric that includes a differentiation between breathing and ventilation. The differentiation between breathing and ventilation is identified, for example, based on expired air. The differentiation is identified, for example, based on a difference between a humidity of expired air and a humidity of inspired air. In various cases, the leak being present is identified based on the difference between the humidity of expired air and the humidity of inspired air being less than a threshold difference. In some examples, a breath of a patient is differentiated from a ventilation based on the difference between the humidity of the expired air and the humidity of the inspired air. In some instances, the user, for example, enable the patient to return to spontaneous respiration based on the difference between the humidity of expired air and the humidity of inspired air being greater than the threshold difference.

For example, because the humidity of inspired air being greater than the humidity of expired air may indicate the medical device 400 is not required by the patient, ceasing the ventilation may enable the patient to continue breathing normally. A difference between the humidity of inspired air and the humidity of expired air being greater than a threshold difference may indicate the medical device 400 is not required by the patient.

In various instances, the leak being present is identified based on a metric that includes an airway fluid-buildup blockage. The airway fluid-buildup blockage is identified, for example, based on the detected humidity that includes a humidity level. A fluid-buildup in an airway, which includes the space in the medical device 400 that is fluidly connected to the airway of the patient, is determined, for example, based on the humidity. The airway fluid-buildup blockage may be present based on the fluid-buildup becoming greater, for example, during long-term ventilation performed by the medical device 400. The fluid-buildup may, for example, cause an issue depending on a hosing material that includes a material of the tubing 414. The airway fluid-buildup blockage is determined, for example, based on the humidity being greater than a humidity threshold. In various implementations, a buildup is identified as being present in the airway, an alert is output, or a combination thereof, based on the airway fluid-buildup blockage. The alert is output, for example, to enable the user to resolve the blocked airway by clearing the blockage to ensure the patient continues to have a suitable airway.

In some examples, any of the metric(s), or any combination thereof, is determined. The leak being present in the airway of the medical device 400 is determined, for example, based on any of the metric(s), or any combination.

In various implementations, coaching data, which is identified based on any of the metric(s), or any combination thereof, being determined, includes ventilation coaching data. The coaching data includes, for example, tidal volume target band data, which includes a tidal volume target band. The coaching data includes, for example, a graph, including a bar graph, a line graph, and so on, or a combination thereof. The graph includes, for example, the tidal volume target band, the current volume of air being integrated from the flow data, or any combination thereof. In some implementations, the graph includes a number of bars being presented on the display 416 to provide feedback. The feedback, for example, includes instantaneous feedback, real-time feedback, and so on. In some cases, the feedback includes a level of air that has been detected by the medical device 400 and that has been administered to the patient. In some implementations, the graph includes a highlighted portion corresponding to a target volume range. The highlighted portion enables the user, for example, to clearly view the target volume range to determine when to stop squeezing the bag 406.

In some examples, the coaching data is presented by the display 416, a display of a sensor that includes any of the air flow sensor 402, the air pressure sensor 404, the CO₂ sensor 422, or a combination thereof.

In some instances, the medical device 400 outputs coaching data, such as audio coaching data or visual coaching data, in response to any data identified by the medical device 400, the monitoring device 102, the intermediary device 106, or any combination thereof, the data including, for example, medical device data, monitoring device data, treating device data, physiological parameter data, sensor status data, feedback data, or any combination thereof. In some instances, the coaching data is used to instruct a caregiver, including a caregiver using the medical device 400 in an ambulance traveling over uneven terrain or a windy road. In some instances, the coaching data is used to instruct a caregiver using the medical device 400 in the field, such as an environment that is noisy, chaotic, etc., where the user may be unable to easily identify treatment to provide to a patient. In those or other instances, the coaching data being used to instruct the caregiver (e.g., the caregiver using the medical device 400 in the ambulance or in the field) where data being identified and/or output by the medical device 400 is not easily received and/or processed by the caregiver for identifying treatment to be provided to the patient. In various cases, the caregiver is a profession caregiver, a caregiver that is person with limited or no medical training, or any other type of caregiver.

In some examples, the coaching data includes different types of coaching data being structure, arranged, formatted, or any combination thereof, for different levels of medical skill of the caregiver. For example, the coaching data is output, in a default mode that is selected by the user, the manufacturer, the distributor, the caregiver, or any combination thereof. The default mode is identified as, and/or set to, an unskilled caregiver level, for example, for instances in which the medical device 400 is likely to be used to an unskilled person. The default mode is set to a skilled caregiver level, for example, for instances in which the medical device 400 is likely to be used by a skilled caregiver. The default mode is set to any of various other levels, for example, for instances in which the medical device 400 is likely to be used by various persons of various levels of skill. Identifying any of the likelihoods includes, for example, identifying whether any of the likelihoods meets or exceeds a threshold likelihood.

In various implementations, the type of the coaching data is modified by the caregiver from the default level to a different level prior any of the coaching data to being output, after a portion of the coaching data is output, and so on, or any combination thereof. The type of the coaching data is selected, for example, based on a coaching data level selection received via user input to the medical device 400. Various portions of the coaching data can include various levels of detail, including, for example, relatively, in-depth data, detailed data, meticulous data, simple data, and so on, or any combination thereof, for the unskilled caregiver level. The various portions of the coaching data can include, for example, relatively, abbreviated data, specific data, complex data, and so on, or any combination thereof, for the skilled caregiver level. Any combinations of the various levels of detail can be utilized as the default level, or modified level, of the coaching data. The various levels of detail can be selectable based on various coaching data detail selections received via user input to the medical device.

The various levels of detail, in some instances, are selectable for different types of medical operations of the medical device 400. For example, different types of unskilled caregiver level coaching data is provided for medical operations likely to be familiar to caregivers, including the unskilled caregiver, the skilled caregiver, or a combination thereof. In some cases, different types of skilled caregiver level coaching data is provided for medical operations likely to be unfamiliar to caregivers, including the unskilled caregiver, the skilled caregiver, or a combination thereof. The coaching data is relatively in-depth, detailed, meticulous, simple, thorough, or any combination thereof, for example, for relatively unique, rare, less-frequently used, operations of the medical device 400. The coaching data is relatively abbreviated, specific, complex, or any combination thereof, for example, for relatively common, known, frequently occurring, operations of the medical device 400.

In some implementations, the coaching data includes instructions for steps of operation of the medical device 400. For example, a portion of the coaching data includes instructions indicating a contraction of the bag 406, a release of the bag 406, a level of the contraction (e.g., a current contraction, a subsequent contraction, any number of subsequent contractions, a group of contractions), a level of the release (e.g., a current release, a subsequent release, any number of subsequent releases, a group of releases), and so on, or any combination thereof.

In various examples, the coaching data includes instructions for an operation including any component of the medical device 400, such as the air flow sensor 402, the air pressure sensor 404, another sensor, the bag 406, the inlet valve 408, the expiratory valve 410, another valve, the mask 412, the tubing 414, the display 416, the indicator 418, the electrode(s) 420, the CO2 sensor, the transceiver 424, and so on, or any combination thereof. For example, the coaching data includes instructions for installing, removing, adjusting, inspecting, replacing, and so on, or any combination thereof, of any of the components. The coaching data, in some examples, including instructions for operating the display 416 to provide user input utilized for performing any operation of the medical device 400.

The coaching data, in some examples, including instructions for how to repair or alleviate the leak between the mask 412 and the patient, for instances with the leak being present. The coaching data, for example, instructs the caregiver to check a portion of the mask identified as being in an area, or being likely to be in an area of, the leak. The coaching data instructs the caregiver how to stop the leak, in some instances, such as by repositioning the medical device 400 (e.g., the mask 412), or any combination thereof. The coaching data instructs the caregiver how to stop the leak, for example, such as by removing an object between the mask 412 and the face of the patient, the object including a medical device object, a patient object, a caregiver object, a foreign object, a liquid, and so on, or any combination thereof.

The coaching data includes, for example, instructions to verify integrity of the medical device 400, how to replace and/or repair any components of the medical device 400, and so on, or any combination thereof. The coaching data, for example, instructs the caregiver how to stop the leak, in some instances, by replacing or repairing a component, such as the mask 412. The coaching data, in some examples, instruct the caregiver to upgrade software of any electrical component of the medical device 400, or disable a component, for example, a component that is malfunctional, that includes outdated software, and the like, or any combination thereof.

In various examples, the coaching data is output in an interactive format by a portion of the coaching data being output, and then, based on user input including a response to an inquiry output via the portion of the coaching data, by a next portion of the coaching data being output, and so on. The interactive format is used to output any portion of the coaching data, for example, by the medical device 400 identifying an instruction or group of instructions to output from among a larger group of available instructions. The identified instructions is identified based on the user input, for example.

In some implementations, the coaching data includes instructions to operate other devices utilized in connection with operation of the medical device 400. For example, the coaching data includes instructions for operating the monitoring device 102, the intermediary device 106, any other device, or any combination thereof. The coaching data, for example, includes instructions for obtaining data to be utilized by the medical device 400 for identifying and/or requesting any other data, such as the medical device data, the monitoring device data, the treating device data, the physiological parameter data, the sensor status data, the feedback data, or any combination thereof.

In various examples, the coaching data, the levels of detail of the coaching data, types of the coaching data, and so on, or any combination thereof, are identified by the medical device 400, the monitoring device 102, the intermediary device 106, or any combination thereof. Any user input utilized to identify and/or modify the coaching data, in some implementations, is received in various instances by the medical device 400, the monitoring device 102, the intermediary device 106, or any combination thereof, and utilized thereby, in a similar way as for the user input received by the medical device, 400, as discussed above.

The coaching data is identified and/or output by the medical device 400, the monitoring device 102, the intermediary device 106, or any combination thereof, in some implementations, in a similar way as any of the data being identified and/or output by the medical device 400, the monitoring device 102, the intermediary device 106, or any combination thereof, as discussed throughout the current disclosure. For example, the coaching data is output as audio data, visual data, or any combination thereof, by any of the devices or any combination of the devices.

In various implementations, feedback data is received by the medical device 100, the monitoring device 102, the intermediary device 106, or any combination thereof. The feedback data includes data based on user input received to the medical device 100, the monitoring device 102, the intermediary device 106, or any combination thereof. The feedback data includes operating instructions, suggestions, treatment response information, treatment, device, and/or sensor status information, and the like, or any combination thereof.

In various implementations, the medical device 400 includes a power supply. The medical device 400, for example, includes a charger connector for transmitting or receiving power, a wireless charging device, or a combination thereof. The wireless charging device, for example, includes an inductive coil for wirelessly receiving power, an inductive coil for wirelessly transmitting power, or a combination thereof. The medical device 400, for example, includes the wireless charging device but not the charger connector. A number of open connections into the medical device 400 being less than a threshold number based on the medical device 400, for example, not including the charger connector enables the medical device 400 to have a relatively higher international protection (“IPX”) waterproof rating than for the medical device 400 including the charger connector. The higher waterproof rating, for example, reduces a likelihood of a medical problem occurring, a problem in operating the medical device 400 occurring, or a combination thereof, based on blood or another fluid being present.

In various implementations, the medical device 400 exchanges communications via the transceiver 424 based on received signal strength indicator (RSSI)-based asset management. The communications include, for example, any data determined by the medical device 400, the monitoring device 102, the intermediary device 106, or any combination thereof, physiological parameter(s). The communications include, for example, the medical device data, the physiological parameter data, the sensor status data, the coaching data, the feedback data, or any combination thereof.

For example, the medical device 400 exchanges communications via the transceiver 424 based on a signal strength. In some examples, the medical device 400 transmits a notification signal to the monitoring device 102, the intermediary device 106, or any combination thereof, based on a signal strength for communications between the medical device 400 and the monitoring device 102, between the medical device 400 and the intermediary device 106 or any combination thereof, being less than a threshold signal strength. The notification signal is used, for example, to indicate the ventilation coaching device being left behind. In some cases, the notification signal is used to present a message on the display of the monitoring device 102, the intermediary device 106, or any combination thereof, to inform the user of the medical device 100 being left behind.

FIG. 5 illustrates an example illustration of a medical device 500 and a modular sensor 502, and an example schematic diagram of an electrical circuit 508 of the modular sensor 502. In some examples, the medical device 500 includes a mask 504. In some implementations, the medical device 500 is the medical device 100, the medical device 200, the medical device 300, or the medical device 400 described above with reference to FIGS. 1-4. In some examples, the mask 504 is the mask 212, the mask 312, or the mask 412 described above with reference to FIGS. 2-4. In some implementations, the sensor 502 is any of the sensor(s) in the medical device 100, or any of the sensors 302 and 304 described above with reference to FIGS. 1 and 3. In some implementations, the modular sensor 502 is implemented similarly to any of the sensors 202 and 204 or any of the sensors 402, 404, and 422 described above with reference to FIGS. 2 and 4, except by being modular.

In some cases, the modular sensor 502 includes a user interface (UI) (e.g., a display, an indicator, a speaker, a haptic feedback device, or a combination thereof). For example, the UI includes a display 506.

In some examples, the modular sensor 502 includes at least one of an electrical circuit 508, at least one electrical component, or a combination thereof. The component(s) include, for example, a processor 510, a power supply 512, a battery charger 514, at least one display driver 516, an SD card 518, a debug interface 520, a sensor interface 522, a wireless device 524, at least one connector 526, at least one physiological parameter sensor 528, at least one other component of any type, or any combination thereof.

The power supply 512 is a battery, for example. In some examples, the circuit 508 is included on a PCB. In some cases, the PCB includes the processor 510, at least one other processor, at least one microprocessor, or any combination thereof. In some examples, the circuit 508 includes the processor 510 integrated within a microprocessor. In some examples, the PCB includes a bus connected to at least one other component of the PCB. The battery charger 514, for example, includes a wireless battery charger including an inductive coil for wirelessly receiving power, an inductive coil for wirelessly transmitting power, or a combination thereof. In some examples, the modular sensor 502 includes at least one connector. Examples of the connector(s) include at least one charger connector, at least one data connector, or a combination thereof. The modular sensor 502, for example, includes the PCB, which includes at least one of the component(s), at least one of the connector(s), or any combination thereof.

Although the power supply 512 may be charged wirelessly, as discussed above in the current disclosure, it is not limited as such. In various cases, the power supply 512 may be charged by a wireless connection, charged by a wired connection, such as via any number of wires and/or cables connected to any number of external power supplies, or any combination thereof.

In various implementations, the electrical circuit 508 includes, or is coupled to, the physiological parameter sensor(s) 528 by the sensor interface 522. At least one type of the physiological parameter sensor(s) 528, for example, corresponds to at least one sensor type of the modular sensor 502. The type(s) of the physiological parameter(s), for example, detected by the physiological parameter sensor(s) correspond to the sensor type(s).

In some examples, the sensor interface 522 optically couples the physiological parameter sensor(s) 528 to at least one of the other component(s). In various examples, the sensor interface 522 couples the physiological parameter sensor(s) 528 to at least one of the other component(s) via the circuit 508. In various instances, conductors and at least one connector connect the physiological parameter sensor(s) 528 to at least one of the other component(s). The conductors, the connector, or the combination thereof, for example, are utilized, additionally or alternatively to the optical interface, to couple the sensor(s) 528 to at least one component of the electrical circuit 508.

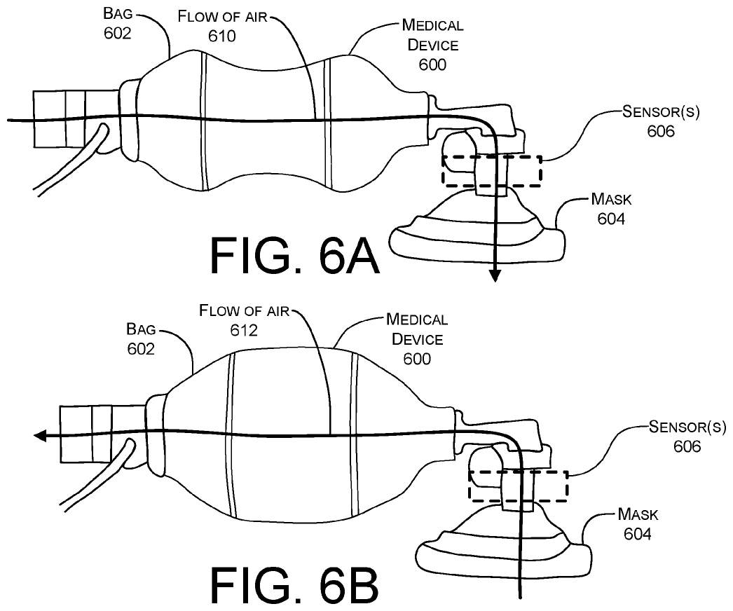

FIGS. 6A and 6B illustrate example illustrations of a medical device 600 being operated to perform ventilation. In some implementations, the medical device 600 is the medical device 100, the medical device 200, the medical device 300, the medical device 400, or the medical device 500 described above with reference to FIGS. 1-5. The medical device 600, for example, includes a bag 602. In some implementations, the bag 602 is the bag in the medical device 100, the bag 206, the bag 306, the bag 406, or the bag in the medical device 500 described above with reference to FIGS. 1-5. The medical device 600, for example, includes a mask 604. In some implementations, the bag 602 is the mask in the medical device 100, the mask 212, the mask 312, the mask 412, or the mask 504 described above with reference to FIGS. 1-5. The medical device 600, for example, includes at least one sensor 606. In some implementations, any of the sensor(s) 606 is any of the sensor(s) in the medical device 300, any of the sensors 202 and 204, any of the sensor(s) in the medical device 300, any of the sensors 402, 404, and 422, or the modular sensor 502 described above with reference to FIGS. 1-5.

As illustrated in FIG. 6A, the bag 602 is contracted. A flow of air 610, for example, is included in the bag 602 based on the bag 602 being contracted. The flow of air 610, for example, is inspiratory air as part of an inspiratory cycle.

As illustrated in FIG. 6B, the bag 602 is contracted. A flow of air 612, for example, is included in the bag 602 based on the bag 602 being contracted. The flow of air 612, for example, is expiratory air as part of an expiratory cycle.

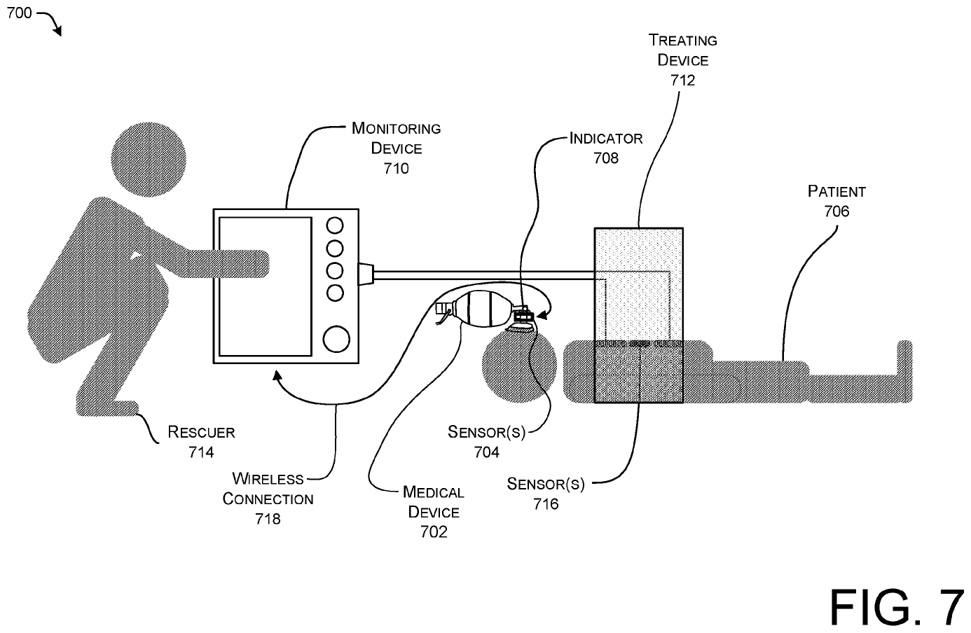

FIG. 7 illustrates an example environment 700 for utilizing a medical device 702 including at least one sensor 704 to treat a patient 706 at a rescue scene. In some implementations, the medical device 702 is the medical device 100, the medical device 200, the medical device 300, the medical device 400, the medical device 500, or the medical device 600 described above with reference to FIGS. 1-6. In some implementations, any of the sensor(s) 704 are any of the sensor(s) in the medical device 100, any of the sensors 202 and 204, any of the sensors 302 and 304, any of the sensors 402, 404, and 422, any of the sensor(s) 528, or any of the sensor(s) 606. Described above with reference to FIGS. 1-6.

In various examples, the medical device 702 includes a user interface (UI) (e.g., a display, an indicator, a speaker, a haptic feedback device, or a combination thereof). The UI, for example includes an indicator 708.

In some examples, the environment 700 includes a monitoring device 710, a treating device 712, or a combination thereof, being operated by a rescuer 714. The treating device 712, for example includes at least one sensor 716. In some examples, the medical devise 702 exchanges communications with the monitoring device 710, the treating device 712, or the combination thereof via a wireless connection 718, a wired connection, or a combination thereof.

For instance, the monitoring device 710 includes a monitor-defibrillator, a medical imaging device, an ultrasound monitor, a standalone ECG monitor, or another type of patient monitor. The monitoring device 710 includes and/or is communicatively coupled to the sensor(s) 716. The sensor(s) 716, for example, is configured to detect at least one physiological parameter of the patient 706. Examples of the physiological parameter(s) include, for instance, an ECG, an impedance, a force administered to the patient 706, a blood pressure, an airway parameter (e.g., a partial pressure of carbon dioxide, a partial pressure of oxygen, a capnograph, an end tidal gas parameter, a flow rate, etc.), a blood oxygenation (e.g., a pulse oximetry value, a regional oximetry value, etc.), an electroencephalogram (EEG), a temperature, a heart sound, a blood flow rate, a physiological geometry (e.g., a shape of a blood vessel, an inner ear shape, etc.), a heart rate, a pulse rate, or the like. For example, the sensor includes at least one of electrodes, a detection circuit, defibrillator pads, a force sensor, a blood pressure cuff, an ultrasound-based blood pressure sensor, an invasive (e.g., intra-arterial) blood pressure sensor (e.g., including a cannula inserted into the patient), a gas sensor (e.g., a carbon dioxide and/or oxygen sensor), a flowmeter, a pulse oximetry sensor a regional oximetry sensor, a thermometer, a microphone, an ultrasound transducer, a medical imaging device (e.g., an ultrasound imaging device), or the like.

In various cases, the monitoring device 710 outputs the physiological parameter(s) to the rescuer 714. For instance, the monitoring device 710 includes a display, a speaker, or haptic feedback device that conveys the physiological parameter(s) to the rescuer 714.

In some examples, the treating device 712 administers a treatment to the patient 706. For example, the treating device 712 includes a monitor-defibrillator, an automated external defibrillator (AED), mechanical chest compression device, a smart bag-valve mask, a ventilator, a heart-lung machine, an intravenous fluid (IV) pump, or the like. Examples of treatments include defibrillation, pacing, cardioversion, administration of chest compressions, administration of oxygen to the airway of the patient 706, movement of air in the airway of the patient, administration of fluids to the patient 706, extracorporeal membrane oxygenation (ECMO), administration of a medication to the patient 706, or the like. In some implementations, the monitoring device 710 is also configured to administer a treatment to the patient 706. Further, in some cases, the treating device 712 is configured to detect one or more physiological parameters of the patient 706.

In some examples, the medical device 702 determines whether a leak is present between a face of the patient 706 and a mask of the medical device 702 based on the physiological parameter(s) detected by the sensor(s) 704. In various examples, the medical device 702, the monitoring device 710, the treating device 712, or a combination thereof, exchange communications including, and/or present, any data determined by the medical device 702, the monitoring device 710, the treating device 712, or a combination thereof. The data presented includes, for example, medical device data, monitoring device data, treating device data, physiological parameter data, sensor status data, coaching data, feedback data, or any combination thereof.

In some examples, the monitoring device 710, the treating device 712, or any combination thereof, determines whether the leak is present and operates in a similar way as the medical device 702. For example, the monitoring device 710, the treating device 712, or any combination thereof outputs an alert indicating that the leak is present.

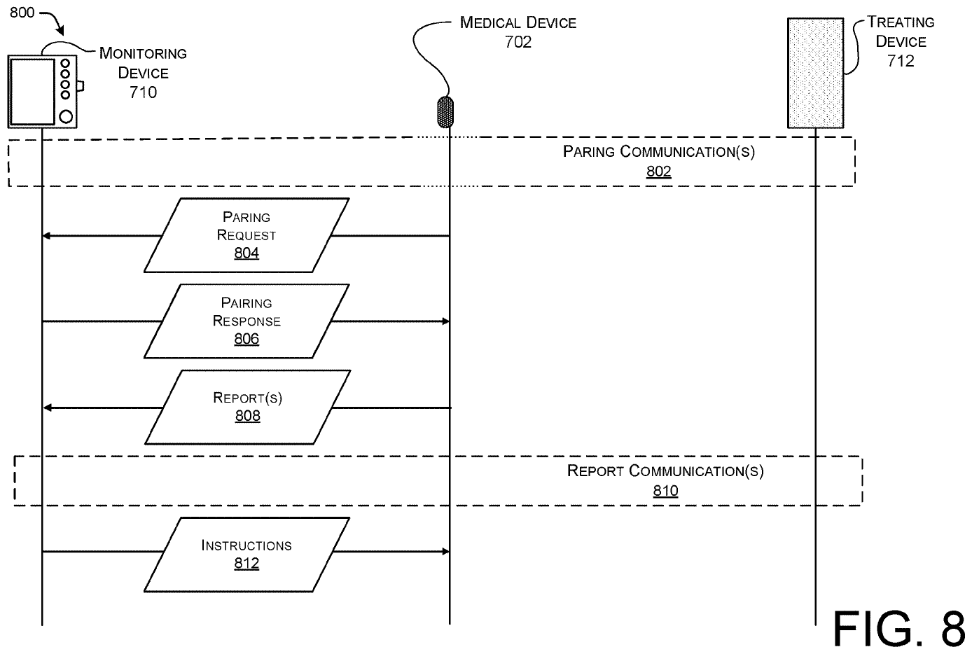

FIG. 8 illustrates an example signal flow 800 for exchanging communications between a medical device 702, a monitoring device 710, and a treating device 712. In some examples, pairing communications 802 are exchanged between the monitoring device 710 and the treating device 712, to pair the monitoring device 710 with the treating device 712.

In some examples, the pairing communications 802 include at least one pairing request transmitted by the monitoring device 710 and to the treating device 712, or vice versa. In some examples, the pairing communications 802 include at least one pairing response received by the monitoring device 710 and from the treating device 712, or vice versa.

In various examples, the medical device 702 transmits a pairing request 804 to the monitoring device 710. In various examples, the medical device 702 receives a pairing response 806 from the monitoring device 710. At least one paring request that includes the pairing request 804, and at least one paring response that includes the pairing response 806, for example, are used to pair the medical device 702 with the monitoring device 710, the treating device 712, or any combination thereof.

In some examples, the medical device 702 exchanges communications with the monitoring device 710. In various examples, the communications include report communications 810. Exchanging the communications, for example, is performed by the medical device 702, the monitoring device 710, the treating device 712, or a combination thereof. Exchanging the communications, for example, includes the medical device 702 transmitting at least one report 808 to the monitoring device 710. The report(s) 808 include, for example, any data determined by the medical device 702. The data transmitted includes, for example, medical device data, physiological parameter data detected by the sensor(s) 704, sensor status data associated with the sensor(s) 704, feedback data, or any combination thereof.

In various instances, exchanging the communications, for example, includes the medical device 702 receiving instructions 812 from the monitoring device 710. The instructions 812 include, for example, any data determined by the monitoring device 710. The data received includes, for example, physiological parameter data detected by the sensor(s) 716, sensor status data associated with the sensor(s) 716, feedback data, or any combination thereof.

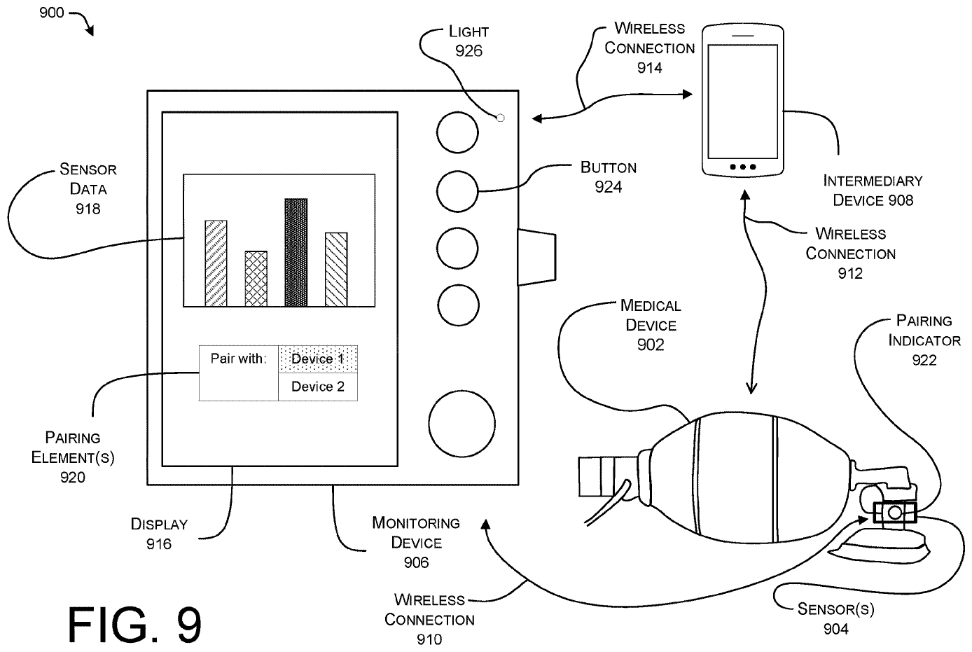

FIG. 9 illustrates an example environment 900 of a medical device 902 including one or more sensors 904 and being interconnected with a monitoring device 906 and with an intermediary device 908. In some implementations, the medical device 902 is the medical device 100, the medical device 200, the medical device 300, the medical device 400, the medical device 500, the medical device 600, or the medical device 702 described above with reference to FIGS. 1-7. In some implementations, the intermediary device 908 is the intermediary device 106. In some implementations, the monitoring device 906 is the monitoring device 710. In some implementations, any of the sensor(s) 904 are any of the sensor(s) in the medical device 100, any of the sensors 202 and 204, any of the sensors 302 and 304, any of the sensors 402, 404, and 422, any of the sensor(s) 528, any of the sensor(s) 606, or any of the sensor(s) 704, described above with reference to FIGS. 1-7.

The medical device 902, for example, exchanges communications with the monitoring device 906 via a wireless connection 910, and with the intermediary device 908 via a wireless connection 912. The monitoring device 906, for example, exchanges communications with the intermediary device 908 via a wireless connection 914. In some implementations, the wireless connection 910 is the wireless connection 108 or the wireless connection 718. In some implementations, the wireless connection 912 is the wireless connection between the medical device 100 and the intermediary device 106. In some implementations, the wireless connection 914 is the wireless connection between the monitoring device 102 and the intermediary device 106.

In various examples, the monitoring device 906 includes a display 916 that presents sensor data 918, at least one paring element 920, or a combination thereof. In some implementations, the display 916 is the display of the monitoring device 102. For example, the display 916 displays data that includes any data as discussed throughout the current disclosure, such as the medical device data, the physiological parameter data, the sensor status data, the coaching data, the feedback data, or any combination thereof.

In some implementations, the medical device 902 includes a display presenting information that is the same as, or different from, information presented by the display 916. In some examples, the information presented by the display in the medical device 902 is in a format that is the same, or different from, as the display 916, and/or with at least one characteristic that is the same as, or different from, at least one corresponding characteristic as the display 916. Implementations, the sensor data 918 includes the physiological parameter data determined based on at least one detected physiological parameter, as described above with reference to FIGS. 1-8.

The sensor status data 918, for example, includes data representing the physiological parameter(s). In some implementations, the sensor status data 918 is any of the data, such as any of the graph(s) as described throughout the current disclosure.

In some examples, the pairing element(s) 920 include a pairing element being activated based on the medical device 902 being paired with the monitoring device 906. In some examples, at least one other pairing element in the pairing element(s) 920 is based on pairing with at least one other medical device (e.g., at least one other BVM device). Pairing, for example, is performed by the pairing request 804 and the pairing response 806.

Any of the pairing element(s) 920, for example, is activated based on the medical device 902 being paired with the monitoring device 906. Activating of a pairing element 920, for example, based on an activation (e.g., a first pairing activation) includes the pairing element 920 being illuminated, the illuminated paring indicator 922 having at least one characteristic (e.g., a color, a blinking level, a shading level, etc.) based on the pairing. The pairing element 920, for example, is deactivated based on the medical device 902 not being paired with the monitoring device 906. In some examples, the pairing element 920 is activated based on an activation (e.g., a second pairing activation) that includes the pairing element 920 being illuminated, the illuminated pairing element 920 having at least one characteristic (e.g., a color, a blinking level, etc.) that is different than at least one corresponding characteristic for the first pairing activation.

In various implementations, any of the pair element(s) 920, a different portion of the display 916, or a combination thereof, include sensor status data. In some implementations, the sensor status data is the modular sensor status data as described above with reference to FIG. 3. In some examples, the display 916 is a touchscreen that receives any user input utilized to control the UI, as discussed throughout the current disclosure.

In some examples, the medical device includes a pairing indicator 922. The pairing indicator 922, for example, is activated based on the medical device 902 being paired with the monitoring device 906. Activating of the paring indicator 922, for example, based on an activation (e.g., a first pairing activation) includes the paring indicator 922 being illuminated, the illuminated paring indicator 922 having at least one characteristic (e.g., a color, a blinking level, etc.) based on the pairing. The pairing indicator 922, for example, is deactivated based on the medical device 902 not being paired with the monitoring device 906. In some examples, the paring indicator 922 is activated based on an activation (e.g., a second pairing activation) that includes the paring indicator 922 being illuminated, the illuminated paring indicator 922 having at least one characteristic (e.g., a color, a blinking level, etc.) that is different than at least one corresponding characteristic for the first pairing activation.

In some examples, the monitoring device 906 includes a button 924 and a light 926. The button 924, the light 926, or a combination thereof, for example, is part of a user interface (UI) of the monitoring device 906. The UI, for example, is the UI of the monitoring device 102. In some examples, the button 924 is used to change the sensor data 918 being presented based on the medical device 902 to other sensor data, or to change one or more other characteristics of the data presented by the display 916. The button 924, for example, receives an input signal from a user when the user activates the button 924. Activation of the button 924, for example, includes the button 924 being pressed, turned, shifted, etc., or any combination thereof. The light 926, for example, indicates a channel and/or a link key associated with wireless communication with the medical device 902.

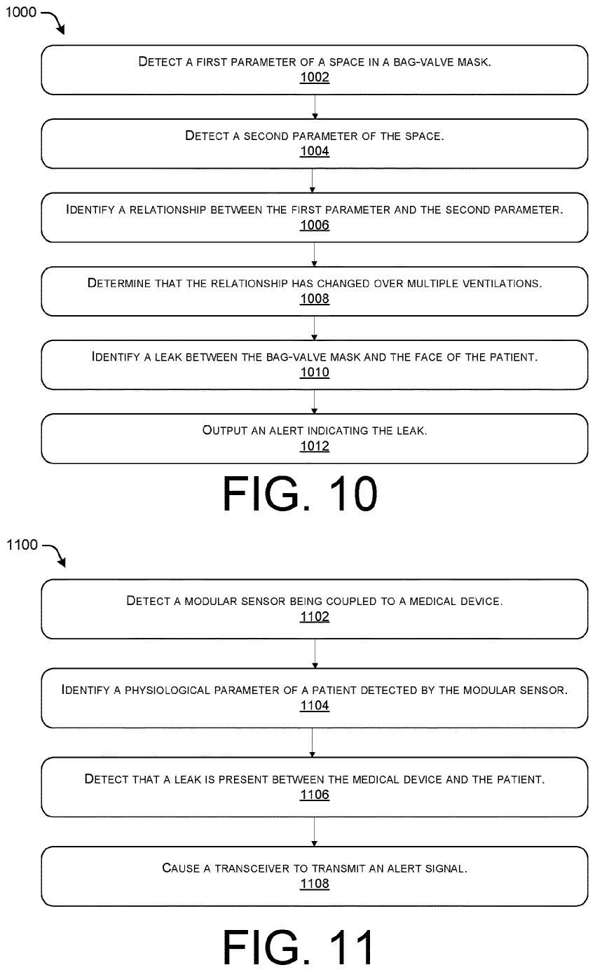

FIG. 10 illustrates an example process 1000 for managing a medical device including an air flow sensor and an air pressure sensor. In some implementations, the medical device is the medical device 100, the medical device 200, the medical device 300, the medical device 400, the medical device 500, the medical device 600, the medical device 702, the medical device 702, or the medical device 902 described above with reference to FIGS. 1-7 and 9.

At 1002, the medical device detects a first parameter of a space in a bag-valve mask. In some examples, the space in the bag-valve mask is fluidly connected to an airway of a patient. For example, the bag-valve mask is disposed on a face of the patient. For example, the first parameter includes a flow rate, an air pressure, a CO2 level, an O2 level, a humidity, a temperature, or the like.

At 1004, the medical device detects a second parameter of the space. For example, the second parameter includes a flow rate, an air pressure, a CO2 level, an O2 level, a humidity, a temperature, or the like.

At 1006, the medical device identifies a relationship between the first parameter and the second parameter. For example, the relationship includes a ratio between the first parameter and the second parameter. In some examples, the relationship includes a relationship between a change in the first parameter between multiple ventilations and a change in the second parameter between the multiple ventilations.

At 1008, the medical device determines that the relationship has changed over multiple ventilations. For example, the multiple ventilations are performed on the patient by the bag-valve mask.

At 1010, the medical device identifies a leak between the bag-valve mask and the face of the patient. For example, the leak is identified based on determining that the relationship has changed over the multiple ventilations.

At 1012, the medical device outputs an alert indicating the leak. For example, the alert is output based on identifying the leak. In some examples, a signal is transmitted to an external device based on the alert or the leak.

FIG. 11 illustrates an example process 1100 for managing a medical device including one or more modular sensors. In some implementations, the medical device is the medical device 100, the medical device 200, the medical device 300, the medical device 400, the medical device 500, the medical device 600, the medical device 702, the medical device 702, or the medical device 902 described above with reference to FIGS. 1-7 and 9.

At 1102, the medical device detects a modular sensor being coupled to a medical device.

At 1104, the medical device identifies a physiological parameter of a patient detected by the modular sensor. In some examples, the physiological parameter is detected in response to the modular sensor being coupled to a medical device. In some examples, detecting the modular sensor being coupled to the medical device includes detecting the modular sensor being coupled to a bag or a mask of the smart bag-valve mask device. In various examples, the physiological parameter includes a flow rate, an air pressure, a CO2 level, an O2 level, a humidity, a temperature, or the like.

At 1106, the medical device detects that a leak is present between the medical device and the patient. The leak is detected, for example, by analyzing the physiological parameter. In some examples, a type of sensor is identified in response to a second modular sensor being coupled to the medical device. The second modular sensor being the identified type of sensor. In various instances, a display is caused to output a second alert indicating that a second physiological parameter is outside of a predetermined parameter range.

At 1108, the medical device causes a transceiver to transmit an alert signal. In some examples, the alert signal indicates that the leak is present between the medical device and the patient. In some examples, the alert signal warns a user to check for the leak, for example, based on the seal not trending better or trending worse, for example, over a number of ventilations (e.g., 2 ventilations, 4 ventilations, etc.).

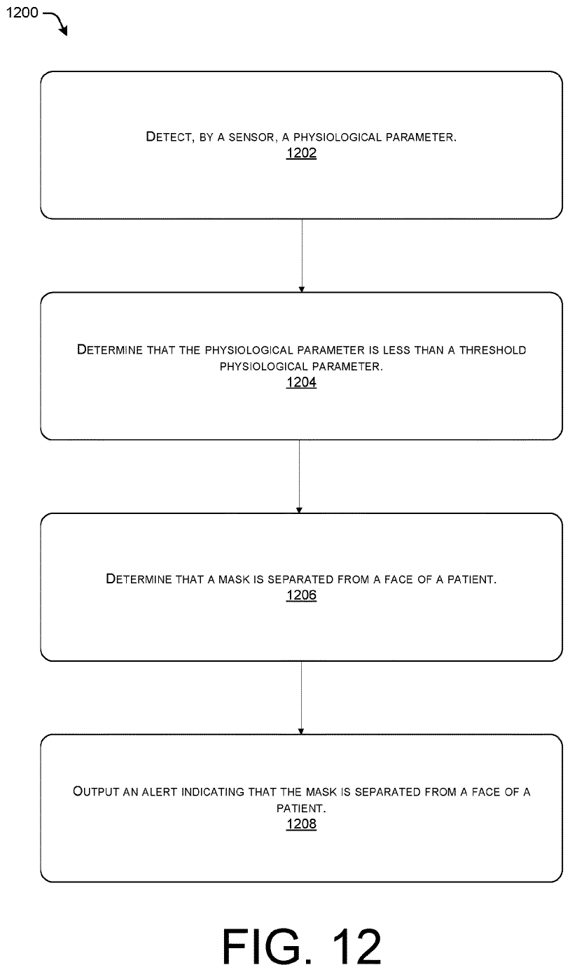

FIG. 12 illustrates an example process 1200 for managing a medical device including an air flow sensor, CO₂ sensor, and an air pressure sensor. In some implementations, the medical device is the medical device 100, the medical device 200, the medical device 300, the medical device 400, the medical device 500, the medical device 600, the medical device 702, the medical device 702, or the medical device 902 described above with reference to FIGS. 1-7 and 9.

At 1202, the medical device detects, by a sensor, a physiological parameter. In various examples, the physiological parameter includes a flow rate, an air pressure, a CO2 level, an O2 level, a humidity, a temperature, or the like.

At 1204, the medical device determines that the physiological parameter is less than a threshold physiological parameter. In some examples, the medical device determines, based on the physiological parameter being detected during a current ventilation, that the mask is separated from the face of a patient by determining the physiological parameter has decreased with respect to a previous physiological parameter detected during a previous ventilation.

At 1206, the medical device determines that a mask is separated from a face of a patient. In some examples, the mask is separated from the face of the patient is determined based on the physiological parameter being less than the threshold physiological parameter,

At 1208, the medical device outputs an alert indicating that the mask is separated from a face of a patient. In various examples, the alert is output visually or audibly as an escalating alert having a first alert level at an initial time being lower than a second alert level at a subsequent time. In various examples, a user interface (UI) identifies an ignore alert selection received via user input to the UI. The alert, for example, is silenced based on the ignore alert selection.

### EXAMPLE CLAUSES

1. A medical device, including: an airway adaptor configured to be connected to an airway of a patient; a first sensor configured to detect a first parameter of a space fluidly connected to the airway adaptor; a second sensor configured to detect a second parameter of the space; a display; and a processor configured to: determine a relationship between the first parameter and the second parameter; based on determining that the relationship has changed over multiple ventilations, identify a leak between the airway adaptor and the airway of the patient; generate an alert indicating the leak; and cause the display to output the alert.
2. The medical device of clause 1, wherein the airway adaptor includes a mask, a supraglottic device, or an endotracheal tube.
3. The medical device of clause 1 or 2, wherein the medical device includes a bag-valve-mask.
4. The medical device of any of clauses 1 to 3, wherein the first parameter includes an air flow rate, wherein the processor is further configured to: determine a first ventilation volume by integrating the air flow rate over a time in which air flows through the space in a first direction; determine a second ventilation volume by integrating the air flow rate over a time in which the air flows through the space in a second direction, the first direction being different than the second direction; wherein identifying the leak includes determining that the first ventilation volume is different than the second ventilation volume.

5. The medical device of clause 4, wherein the medical device includes a ventilation device.

6. The medical device of any of clauses 1 to 5, further including: a gas source fluidly coupled to the space, the gas source being configured to push air through the space.

7. The medical device of clause 6, wherein the gas source includes a bag, a mechanical ventilator, or a gas tank.
8. The medical device of any of clauses 1 to 7, wherein the first parameter includes an air pressure, and wherein the processor is further configured to generate the alert indicating, at a time associated with a compression level of an air bag of the medical device being greater than or equal to a threshold compression level, the air pressure being less than a predetermined air pressure level.
9. The medical device of any of clauses 1 to 8, wherein the first parameter includes a humidity level, and wherein the processor is further configured to identify a physiological characteristic at a time associated with a compression level of an air bag of the medical device being greater than or equal to a threshold compression level, the physiological characteristic representing the humidity level being less than a predetermined humidity level.
10. The medical device of any of clauses 1 to 9, further including: a third sensor coupled to a rim of the airway adaptor, the sensor being configured to detect a first value at a first time and a second value at a second time, the first value including a first electrical resistance or capacitance, the second value including a second electrical resistance or capacitance, the first time occurring when air is traveling into the space, the second time occurring when air is traveling out of the space, wherein the processor is further configured to: determine that a difference between the first value and the second value is greater than or equal to a threshold difference; and in response to determining that the difference between the first value and the second value is greater than or equal to the threshold difference, generate a third alert.
11. The medical device of any of clauses 1 to 10, wherein the first parameter includes an air flow rate, the second parameter includes an O₂ level, wherein the processor is further configured to: determine that the relationship between the first parameter and the second parameter at a first time is an analogous relationship; and wherein determining that the relationship has changed over multiple ventilations includes determining that the relationship between the first parameter and the second parameter at a second time is no longer an analogous relationship.
12. The medical device of any of clauses 1 to 11, wherein the first parameter includes a CO₂ level and the second parameter includes an O₂ level, wherein the processor is further configured to: determine that the relationship between the first parameter and the second parameter at a first time is an inverse relationship, and wherein determining that the relationship has changed over multiple ventilations includes determining that the relationship between the first parameter and the second parameter at a second time is no longer an inverse relationship.
13. The medical device of any of clauses 1 to 12, wherein the first parameter includes an air flow rate, and the second parameter includes an O₂ level, and wherein the processor is configured to cause the display to output numeric values representing the air flow rate, the O₂ level, and a pressure of air in the space.
14. A smart bag-valve mask device, including: a mask configured to be in contact with a face of a patient; a bag fluidly connected to the mask, the mask and the bag enclosing a space that is fluidly connected to an airway of the patient, a first ventilation occurring when the bag is contracted and released a first time, a second ventilation occurring when the bag is contracted and released a second time; a first sensor configured to detect a flow of air in the space; a second sensor configured to detect a pressure in the space; a display configured to visually output an air flow waveform representing the flow over time, and an air pressure waveform representing the pressure over time; and a processor configured to: determine a first ventilation volume by analyzing the flow of air during the first ventilation; determine a second ventilation volume by analyzing the flow of air during the second ventilation; identify a first ratio between the first ventilation volume and the pressure during the first ventilation; identify a second ratio between the second ventilation volume and the pressure during the second ventilation; determine a discrepancy between the first ratio and the second ratio; in response to determining the discrepancy between the first ratio and the second ratio, determine that a leak is present between the mask and the face of the patient; and cause the display to output an alert indicating that the leak is present between the mask and the face of the patient.
15. The smart bag-valve mask device of clause 14, the alert being a first alert, the smart bag-valve mask device further including: a third sensor configured to detect a physiological parameter in the space, the physiological parameter including a partial pressure of CO_{2,} a temperature, or a humidity, wherein the display is further configured to output a physiological parameter waveform representing the physiological parameter over time, and wherein the processor is further configured to: determine that the leak has greater than a threshold severity by analyzing the physiological parameter; and in response to determining that the leak has greater than the threshold severity: increase an intensity of the first alert; or cause the display to output a second alert indicating that the leak has greater than the threshold severity.
16. The smart bag-valve mask device of clause 14 or 15, the alert being a first alert, the smart bag-valve mask device further including: an electrode disposed on an edge of the mask and configured to detect an electrical signal, wherein the processor is further configured to: detect a separation between the edge of the mask and the face of the patient by analyzing the electrical signal; and in response to detecting the separation between the edge of the mask and the face of the patient: increase an intensity of the first alert; or cause the display to output a second alert indicating that the mask is separated from the face of the patient.
17. The smart bag-valve mask device of any of clauses 14 to 16, the alert being a first alert, the smart bag-valve mask device further including: an accelerometer configured to detect a movement of a chest of the patient, wherein the processor is further configured to: detect a separation between the mask and the face of the patient by analyzing the movement of the chest of the patient in view of the flow or pressure; and in response to detecting the separation between the mask and the face of the patient: increase an intensity of the first alert; or cause the display to output a second alert indicating that the mask is separated from the face of the patient.
18. A smart bag-valve mask device, including: a mask configured to be in contact with a face of a patient; a bag fluidly connected to the mask, the mask and the bag enclosing a space that is fluidly connected to an airway of the patient; a first sensor configured to detect a flow of air in the space; a second sensor configured to detect a pressure in the space; a third sensor configured to detect a level of CO₂; and a processor configured to: in response to detecting the flow, the pressure, and the level of CO₂, determine that a leak is present between the mask and the face of the patient; and cause a display to output an alert indicating that the leak is present between the mask and the face of the patient.
19. A smart bag-valve mask device, including: a mask configured to be in contact with a face of a patient; a bag fluidly connected to the mask, the mask and the bag enclosing a space that is fluidly connected to an airway of the patient, a first ventilation occurring when the bag is contracted and released a first time, a second ventilation occurring when the bag is contracted and released a second time; a first sensor configured to detect a flow of air in the space; a second sensor configured to detect a pressure in the space; a transceiver; and a processor configured to: determine a first ventilation volume by analyzing the flow of air during the first ventilation; determine a second ventilation volume by analyzing the flow of air during the second ventilation; identify a first ratio between the first ventilation volume and the pressure during the first ventilation; identify a second ratio between the second ventilation volume and the pressure during the second ventilation; determine a discrepancy between the first ratio and the second ratio; in response to determining the discrepancy between the first ratio and the second ratio, determine that a leak is present between the mask and the face of the patient; and cause the transceiver to transmit an alert indicating that the leak is present between the mask and the face of the patient.
20. A smart bag-valve mask device, including: a mask configured to be in contact with a face of a patient; a bag fluidly connected to the mask, the mask and the bag enclosing a space that is fluidly connected to an airway of the patient, a ventilation occurring when the bag is contracted and released; a sensor configured to detect a flow of air in the space, the flow over time being utilized to identify a volume of the air; a display configured to visually output an air flow value representing the flow, and a volume value representing the volume of the air; and a processor configured to: determine an inhalation volume by analyzing the volume of the air when the bag is contracted; determine an exhalation volume by analyzing the volume of the air when the bag is released; identify a discrepancy between the inhalation volume and the exhalation volume; in response to identifying the discrepancy between the inhalation volume and the exhalation volume, determine that a leak is present between the mask and the face of the patient; and cause the display to output an alert indicating that the leak is present between the mask and the face of the patient.
21. A method, including: detecting a first parameter of a space in a bag-valve mask that is fluidly connected to an airway of a patient and that is disposed on a face of the patient; detecting a second parameter of the space; identifying a relationship between the first parameter and the second parameter; determining that the relationship has changed over multiple ventilations performed on the patient by the bag-valve mask; based on determining that the relationship has changed over the multiple ventilations, identifying a leak between the bag-valve mask and the face of the patient; and based on identifying the leak, outputting an alert indicating the leak.
22. The method of clause 21, wherein the first parameter includes an air flow rate, further including: integrating the air flow rate over time; and identifying a result of the air flow rate being integrated over time as a ventilation volume, and wherein determining the leak includes determining the leak based on the ventilation volume.
23. The method of clause 21 or 22, wherein the first parameter includes an air flow rate, the second parameter includes an air pressure level, and the relationship includes an analogous relationship.
24. The method of any of clauses 21 to 23, wherein the first parameter includes an air pressure level, the second parameter includes a CO₂ level, the CO₂ level includes a partial pressure of CO₂ or an EtCO₂, and the relationship includes an analogous relationship.
25. The method of any of clauses 21 to 24, wherein the first parameter includes a CO₂ level, the relationship includes a first relationship, and the alert includes a first alert identify at a first time, the method further including: identifying a third parameter, the third parameter including an O₂ level of air in the airway; identifying a second relationship between the CO₂ level and the O₂ level; determining that the second relationship has changed over the multiple ventilations; based on determining that the relationship has changed over the multiple ventilations, identifying a second leak at a second time between the bag-valve mask and the face of the patient; and based on identifying the second leak at the second time, outputting an alert indicating the leak.
26. The method of any of clauses 21 to 25, wherein the first parameter includes a CO₂ level, the CO₂ level includes a partial pressure of CO₂, the relationship includes a first relationship, and the alert includes a first alert, the method further including: identifying a third parameter; identifying a second relationship between the CO₂ level and the third parameter, the third parameter including a humidity level or a temperature level of air in the airway; determining that the second relationship has changed over the multiple ventilations; based on determining that the relationship has changed over the multiple ventilations, identifying the CO₂ level is outside of a predetermined CO₂ level range; and based on identifying the CO₂ level being outside of a predetermined CO₂ level range, outputting an alert indicating the leak.
27. The method of any of clauses 21 to 26, wherein causing a display to output the alert further includes: causing the display to output the alert visually or audibly as an escalating alert having a first alert level at an initial time being lower than a second alert level at a subsequent time.
28. The method of any of clauses 21 to 27, wherein the alert includes a first alert, further including: identifying a first electrode and a second electrode attached to a rim of the bag-valve mask, an electrical resistance level; and causing a display of the bag-valve mask to output a second alert warning to check for the leak, the second alert identifying the electrical resistance level being less than or equal to a threshold electrical resistance level.
29. The method of any of clauses 21 to 28, wherein the alert includes a first alert, further including: identifying, via a first sensor and a second sensor attached to a rim of the bag-valve mask, an electrical resistance level; and causing a display of the bag-valve mask to output a second alert warning to check for the leak, the second alert identifying the electrical resistance level being less than or equal to a threshold electrical resistance level.
30. The method of any of clauses 21 to 29, wherein the alert includes a first alert, the method further including: identifying, via a capacitive sensor attached to a rim of the bag-valve mask, a capacitance level; and causing a display of the bag-valve mask to output a second alert warning to check for the leak, the second alert identifying the capacitance level being less than or equal to a threshold capacitance level.
31. The method of any of clauses 21 to 30, further including: outputting, audibly or visibly, coaching instructions for steps of operation of the bag-valve mask.
32. The method of any of clauses 21 to 31, further including: identifying a blood oxygenation; and comparing the blood oxygenation to a threshold, wherein identifying the leak is further based on comparing the blood oxygenation to the threshold.
33. The method of clause 32, wherein identifying the blood oxygenation includes receiving, from an oximetry device, an indication of the blood oxygenation.
34. The method of any of clauses 21 to 33, wherein the first parameter includes a volume of air flowing from the bag-valve mask into the airway, wherein the second parameter includes a pressure of air in the bag-valve mask, and wherein the method further includes: determining a lung compliance based on a change in the volume of air and a change in the pressure of air.
35. A medical device, including: a first sensor configured to detect a first parameter of a space in a bag-valve mask that is fluidly connected to an airway of a patient; a second sensor configured to detect a second parameter of the space; a display; and a processor configured to: determine a relationship between the first parameter and the second parameter; based on determining that the relationship has changed over multiple ventilations, identify a leak between the bag-valve mask and a face of the patient; generate an alert indicating the leak; and cause the display to output the alert.
36. The medical device of clause 35, wherein the first parameter includes an air flow rate, wherein the processor is further configured to: integrate the air flow rate over time; and identify a result of the air flow rate being integrated over time as a ventilation volume, and wherein identifying the leak includes identifying the leak based on the ventilation volume.
37. The medical device of clause 35 or 36, wherein the first parameter includes an air pressure, and wherein the processor is configured to generate the alert in response to determining that, at a time: a compression level of an air bag of the medical device is greater than or equal to a threshold compression level, and the air pressure is less than a predetermined air pressure level.
38. The medical device of any of clauses 35 to 37, wherein the first parameter includes a humidity level, and wherein the processor is further configured to identify a physiological characteristic at a time associated with a compression level of an air bag of the medical device being greater than or equal to a threshold compression level, the physiological characteristic representing the humidity level being less than a predetermined humidity level.
39. The medical device of any of clauses 35 to 38, further including: a rim; and a sensor attached to the rim, the sensor identifying an electrical resistance or a capacitance, wherein the processor is further configured to: generate a third alert indicating a difference between a first sensor value of the sensor and a second sensor value of the sensor being greater than or equal to a threshold difference, the first sensor value identifying the electrical resistance or the capacitance at a first time of air in the space travelling into the medical device, the second sensor value identifying the electrical resistance or the capacitance at a second time of air in the space travelling out of the medical device.
40. The medical device of any of clauses 35 to 39, wherein the first parameter includes an air flow rate, the second parameter includes an O2 level, and wherein the processor is further configured to: determine that the relationship between the first parameter and the second parameter is an analogous relationship; and based on determining that the relationship has changed over the multiple ventilations to not being the analogous relationship, identify the leak.
41. The medical device of any of clauses 35 to 40, wherein the first parameter includes a CO₂ level, the CO₂ level including a partial pressure of CO₂, and the second parameter includes an O₂ level, and wherein the processor is further configured to: determine that the relationship between the first parameter and the second parameter is an inverse relationship; and based on determining that the relationship has changed over the multiple ventilations to not being the inverse relationship, identify the leak.
42. A smart bag-valve mask device, including: a mask configured to be in contact with a face of a patient; a bag fluidly connected to the mask, the mask and the bag enclosing a space that is fluidly connected to an airway of the patient; a sensor connector; a modular sensor being coupled to the mask, the bag, or the sensor connector, the modular sensor being configured to detect a physiological parameter of the patient; a display configured to visually output a waveform representing the physiological parameter over time; and a processor configured to: identify a type of sensor in response to the modular sensor being coupled to the mask, the bag, or the sensor connector; cause the modular sensor to detect the physiological parameter in response to the type of sensor being identified; detect, by analyzing the physiological parameter, a leak between the mask and the face of the patient; and cause the display to output an alert indicating the leak between the mask and the face of the patient.
43. The smart bag-valve mask device of clause 42, the modular sensor being coupled to the sensor connector, the smart bag-valve mask further including: a transceiver, wherein the processor is further configured to: cause the transceiver to transmit a status signal indicating the modular sensor being coupled to the sensor connector; and
cause the transceiver to transmit an alert signal in response to the physiological parameter indicating the leak between the mask and the face of the patient.
44. The smart bag-valve mask device of clause 42 or 43, the modular sensor including a first modular sensor, the sensor connector including a first sensor connector, the type of sensor including a first type of sensor; the smart bag-valve mask further including: a second modular sensor; a second sensor connector; and a transceiver, wherein the processor is further configured to: identify a second type of sensor in response to the second modular sensor being coupled to the mask, the bag, or the second sensor connector; identify a second physiological parameter detected by the second modular sensor in response to the second type of sensor being identified; and cause the transceiver to transmit a status signal including the first type of sensor and the second type of sensor.
45. A method, including: detecting a modular sensor being coupled to a medical device; identifying a physiological parameter of a patient detected by the modular sensor in response to the modular sensor being coupled to a medical device; detecting, by analyzing the physiological parameter, that a leak is present between the medical device and the patient; and causing a transceiver to transmit an alert signal indicating that the leak is present between the medical device and the patient.
46. The method of clause 45, wherein the medical device includes a smart bag-valve mask device, and wherein detecting the modular sensor being coupled to the medical device includes detecting the modular sensor being coupled to a bag or a mask of the smart bag-valve mask device.
47. The method of clause 45 or 46, the modular sensor including a first modular sensor, the alert signal including a first alert signal, the leak including a first leak, the method further including: identifying a type of sensor in response to a second modular sensor being coupled to the medical device; detecting a second physiological parameter in response to the type of sensor being identified; and causing the transceiver to transmit a second alert signal indicating that the second physiological parameter is outside of a predetermined parameter range.
48. The method of any of clauses 45 to 47, the modular sensor including a first modular sensor, the alert signal including a first alert signal, the leak including a first leak, the method further including: identifying a type of sensor in response to a second modular sensor being coupled to the medical device; causing a display to output a second alert indicating that a second physiological parameter is outside of a predetermined parameter range.
49. The method of any of clauses 45 to 48, further including: causing a display to output an identifier representing the physiological parameter; identifying the modular sensor being decoupled from the medical device; and causing the display to cease outputting of the identifier in response to the modular sensor being decoupled from the medical device.
50. The method of any of clauses 45 to 49, further including: causing a display to output a first notification of the modular sensor being coupled to the medical device; identifying the modular sensor being decoupled from the medical device; and causing a display to output a second notification of the modular sensor being decoupled to the medical device.
51. The method of any of clauses 45 to 50, the medical device being a smart bag-valve mask device, wherein detecting the physiological parameter further includes detecting the physiological parameter in response to the modular sensor being coupled to a bag or a mask of the medical device; identifying the modular sensor being decoupled from the bag or the mask; and causing a transceiver to transmit an alert signal in response to the modular sensor being decoupled from the bag or the mask.
52. The method of any of clauses 45 to 51, the modular sensor including a first modular sensor, the physiological parameter including a first physiological parameter, wherein detecting the modular sensor being coupled to the medical device further includes activating the first modular sensor to detect the first physiological parameter in response to the first modular sensor being coupled to the medical device, and wherein detecting the first physiological parameter further includes detecting the first physiological parameter in response to the modular sensor being activated, further including: activating a second modular sensor to detect a second physiological parameter in response to the second modular sensor being coupled to the medical device; and deactivating the first modular sensor in response to the second modular sensor being activated.
53. The method of any of clauses 45 to 52, wherein the physiological parameter includes an air flow level, an air pressure, a CO₂ level, an O₂ level, a humidity level, a temperature level, an electrical resistance level, a capacitance level, a bag compression level, or a different type of physiological parameter.
54. The method of any of clauses 45 to 53, wherein detecting the modular sensor further includes: identifying a type of sensor in response to a modular sensor being coupled to a medical device; and activating the modular sensor to detect the physiological parameter in response to the type of sensor.
55. A medical device, including: a modular sensor configured to identify a physiological parameter associated with a patient; a transceiver; and a processor configured to: detect, by analyzing the physiological parameter, that a leak is present between the medical device and the patient; and cause the transceiver to output an alert signal indicating that the leak is present between the medical device and the patient.
56. The medical device of clause 55, wherein the modular sensor is a first modular sensor, the physiological parameter is different from a second physiological parameter detected by a second modular sensor, wherein causing the transceiver to output the alert signal further includes: causing the transceiver to output the alert signal indicating that the second physiological parameter is outside of a predetermined parameter range.
57. The medical device of clause 55 or 56, wherein the physiological parameter is a first physiological parameter including an air flow, an air pressure level, a CO₂ level, an O₂ level, a humidity level, a temperature level, an electrical resistance level, a capacitance level, a bag compression level, or an other type of physiological parameter, wherein a second physiological parameter includes the air flow, the air pressure level, the CO₂ level, the O2 level, the humidity level, the temperature level, the electrical resistance level, the capacitance level, the bag compression level, or the other type of physiological parameter, wherein a first type of the first physiological parameter is different from a second type of the second physiological parameter, and wherein the processor is configured to detect that the leak is present between the medical device and the patient further by analyzing the second physiological parameter.
58. The medical device of any of clauses 55 to 57, wherein the physiological parameter is a first physiological parameter, the processor further configured to: cause a display to output a first identifier representing the first physiological parameter; and cause the display to output a second identifier representing a second physiological parameter.
59. The medical device of any of clauses 55 to 58, wherein the processor is further configured to: identify the modular sensor being decoupled from the medical device; and cause the transceiver to output a status signal indicating the modular sensor being decoupled from the medical device.
60. The medical device of any of clauses 55 to 59, the modular sensor being a first modular sensor, the processor further configured to: identify a second modular sensor being coupled to the medical device; and cause the transceiver to output a status signal indicating the second modular sensor being coupled to the medical device.
61. The medical device of any of clauses 55 to 60, wherein the processor is further configured to: identify the modular sensor being decoupled from the medical device; and cause a display to output a notification of the modular sensor being decoupled from the medical device.
62. The medical device of any of clauses 55 to 61, further including: a power supply; and a power cable utilized to charge the power supply.
63. The medical device of any of clauses 55 to 62, further including: a haptic device, wherein, in response to detecting that the leak is present, the processor is further configured to cause the haptic device to provide haptic feedback.
64. The medical device of any of clauses 55 to 63, wherein the processor is further configured to: identify a chest compression altitude parameter received via the transceiver; and detect, by analyzing the physiological parameter and the chest compression altitude parameter, that the leak is present between the medical device and the patient.
65. A medical device, including: a modular sensor including a first sensor and a second sensor, the first sensor configured to identify a first physiological parameter associated with a patient, the second sensor configured to identify a second physiological parameter associated with the patient; a transceiver; and a processor configured to: detect, by analyzing the first physiological parameter or the second physiological parameter, that a leak is present between the medical device and the patient; and cause the transceiver to output an alert signal indicating that the leak is present between the medical device and the patient.
66. A medical device, including: a modular sensor configured to identify a physiological parameter associated with a patient; a transceiver configured to transmit and receive data; and a processor configured to: cause the transceiver to receive patient data; detect, by analyzing the physiological parameter in response to receiving the patient data, that a leak is present between the medical device and the patient; and cause the transceiver to output an alert signal indicating that the leak is present between the medical device and the patient.
67. A device, including: a mask configured to be in contact with a face of a patient; a bag; a flow rate sensor coupled between the mask and the bag and configured to detect a flow rate of air flowing between the mask and the bag; a CO₂ sensor coupled between the mask and the bag and configured to detect a CO₂ level in the air; an air pressure sensor coupled between the mask and the bag, the air pressure sensor being configured to detect a pressure of the air; a transceiver; a display; and a processor configured to: determine, based on the flow rate, the CO₂ level, and the pressure, that a leak is present between the face and the mask; and in response determining that the leak is present between the face and the mask: cause the transceiver to transmit a signal indicating the leak; and
cause the display to output an alert indicating the leak.
68. The device of clause 67, wherein the processor is configured to determine, based on the flow rate, the CO₂level, and the pressure, that the leak is present between the face and the mask by determining that the flow rate, the CO₂ level, or the pressure is less than a predetermined parameter level.
69. The device of clause 67 or 68, wherein the processor is configured to determine, based on the flow rate, the CO₂ level, and the pressure being detected during a current ventilation, that the leak is present between the face and the mask by determining that the flow rate, the CO₂ level, or the pressure has decreased with respect to a previous flow rate, a previous CO₂ level, or a previous pressure detected during a previous ventilation.
70. The device of any of clauses 67 to 69, wherein the processor is further configured to: determine, based on the flow rate, the CO₂ level, and the pressure, that the leak has ceased; and in response to determining that the leak has ceased, cause the display to cease outputting the alert.
71. The device of any of clauses 67 to 70, wherein the processor is further configured to: receive, at a receiver coil, an electromagnetic signal from an external device, the electromagnetic signal inducing a current in the receiver coil;
store, in a capacitor or battery, energy generated by the current; and supply the energy to the flow rate sensor, the CO₂ sensor, or the air pressure sensor.
72. A method, including: detecting, by a sensor, a physiological parameter; determining that the physiological parameter is less than a threshold physiological parameter; determining, based on the physiological parameter being less than the threshold physiological parameter, that a mask is separated from a face of a patient; and outputting an alert indicating that the mask is separated from a face of a patient.
73. The method of clause 72, wherein the threshold physiological parameter includes a first threshold physiological parameter, wherein determining that the physiological parameter is less than the threshold physiological parameter further includes determining that the physiological parameter is less than the first threshold physiological parameter and greater than a second threshold physiological parameter, and wherein determining that the mask is separated from the face of the patient further includes determining, based on the physiological parameter being less than the first threshold physiological parameter and greater than the second threshold physiological parameter, that the mask is separated from the face of the patient.
74. The method of clause 72 or 73, wherein the physiological parameter includes a flow rate of air flowing between the mask and a bag, a CO₂ level of the air, or a pressure of the air; and wherein outputting the alert includes: determining the physiological parameter is less than a first threshold or greater than a second threshold; and causing a transceiver to wirelessly transmit an alert signal to an external device.
75. The method of any of clauses 72 to 74, wherein outputting the alert includes: determining that the physiological parameter is less than a first threshold or greater than a second threshold; and outputting, over a wired interface, an alert.
76. The method of any of clauses 72 to 75, wherein outputting the alert includes: causing a speaker to output an audible signal including the alert.
77. The method of any of clauses 72 to 76, wherein the physiological parameter is a flow rate, a CO₂ level, or a pressure, and wherein determining, based on the physiological parameter being less than the threshold physiological parameter, that the mask is separated from the face of the patient includes determining that the flow rate, the CO₂ level, or the pressure has decreased with respect to a previous flow rate, a previous CO₂ level, or a previous pressure detected during a previous ventilation.
78. The method any of clauses 72 to 77, wherein the physiological parameter is a flow rate, further including:
   detecting, by a second sensor, a CO₂ level, and wherein determining, based on the physiological parameter being less than the threshold physiological parameter, that the mask is separated from the face of the patient includes determining that the flow rate with respect to a previous flow rate detected during a previous ventilation, the determining of the mask being separated from the face of the patient being further based on the CO₂ level having decreased with respect to a previous CO₂ level detected during the previous ventilation.
79. The method any of clauses 72 to 78, wherein the physiological parameter is a humidity level, and wherein determining, based on the physiological parameter being less than the threshold physiological parameter, that the mask is separated from the face of the patient includes determining the humidity level has decreased with respect to a previous humidity level detected during a previous ventilation.
80. The method any of clauses 72 to 79, wherein the physiological parameter is a temperature level, and wherein determining, based on the physiological parameter being less than the threshold physiological parameter, that the mask is separated from the face of the patient includes determining the temperature level has decreased with respect to a previous temperature level detected during a previous ventilation.
81. The method any of clauses 72 to 80, further including: determining, based on the physiological parameter, that a leak between the mask and face has ceased; and in response to determining that the leak has ceased, cause a display to cease outputting the alert.
82. The method any of clauses 72 to 81, further including: receiving, at a receiver coil, an electromagnetic signal from an external device, the electromagnetic signal inducing a current in the receiver coil; storing, in a capacitor or battery, energy generated by the current; and supplying the energy to a sensor configured to detect the physiological parameter.
83. A medical device, including: a flow rate sensor; a CO₂ sensor; an air pressure sensor; a transceiver; a display; and a processor configured to: determine, based on a flow rate detected by the flow rate sensor, a CO₂ level detected by the CO₂ sensor, or an air pressure detected by the air pressure sensor, that a leak is present between a face and a mask; and in response to determining that the leak is present between the face and the mask: cause the transceiver to transmit a signal indicating the leak; and cause the display to output an alert indicating the leak.
84. The medical device of clause 83, wherein the processor is configured to determine, based on the flow rate, the CO₂ level, and the air pressure, that the leak is present between the face and the mask by determining the flow rate, the CO₂ level, or the air pressure is less than a predetermined parameter level.
85. The medical device of clause 83 or 84, wherein the processor is configured to determine, based on the flow rate, the CO₂ level, and the air pressure being detected during a current ventilation, that the leak is present between the face and the mask by determining the flow rate, the CO₂ level, or the air pressure has decreased with respect to a previous flow rate, a previous CO₂ level, or a previous air pressure detected during a previous ventilation.
86. The medical device of any of clauses 83 to 85, wherein the processor is further configured to: determine, based on the flow rate, the CO₂ level, and the air pressure, that the leak has ceased; and in response to determining that the leak has ceased, cause the display to cease outputting the alert.
87. The medical device of any of clauses 83 to 86, wherein the processor is further configured to: receive, at a receiver coil, an electromagnetic signal from an external device, the electromagnetic signal inducing a current in the receiver coil; store, in a capacitor or battery, energy generated by the current; and supply the energy to the flow rate sensor, the CO₂ sensor, or the air pressure sensor.
88. The medical device of any of clauses 83 to 87, wherein the processor is configured to determine, based on the flow rate, the CO2 level, and the air pressure being detected during a current ventilation, that the mask is separated from the face of a patient by determining that the flow rate, the CO₂ level, or the air pressure has decreased with respect to a previous flow rate, a previous CO₂ level, or a previous air pressure detected during a previous ventilation.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1-88. (canceled)

89. A smart bag-valve mask device, comprising:
a mask configured to be in contact with a face of a patient;
a bag fluidly connected to the mask, the mask and the bag enclosing a space that is fluidly connected to an airway of the patient;
a first sensor configured to detect a flow of air in the space;
a second sensor configured to detect a pressure in the space;
a display configured to visually output an air flow waveform representing the flow of air over time, and an air pressure waveform representing the pressure over time; and
a processor configured to:
determine a first ventilation volume by analyzing the flow of air during a first ventilation occurring when the bag is contracted and released a first time;
determine a second ventilation volume by analyzing the flow of air during the second ventilation occurring when the bag is contracted and released a second time;
identify a first ratio between the first ventilation volume and the pressure during the first ventilation;
identify a second ratio between the second ventilation volume and the pressure during the second ventilation;
determine a discrepancy between the first ratio and the second ratio;
in response to determining the discrepancy between the first ratio and the second ratio, determine that a leak is present between the mask and the face of the patient; and
cause the display to output an alert indicating that the leak is present.

90. The smart bag-valve mask device of claim 89, the alert being a first alert, the smart bag-valve mask device further comprising:
a third sensor configured to detect a physiological parameter in the space, the physiological parameter comprising a partial pressure of CO₂, a temperature, or a humidity,
wherein the display is further configured to output a physiological parameter waveform representing the physiological parameter over time, and wherein the processor is further configured to:
determine that the leak has greater than a threshold severity by analyzing the physiological parameter; and
in response to determining that the leak has greater than the threshold severity:
increase an intensity of the first alert; or cause the display to output a second alert indicating that the leak has greater than the threshold severity.

91. The smart bag-valve mask device of claim 89, the alert being a first alert, the smart bag-valve mask device further comprising:
an electrode disposed on an edge of the mask and configured to detect an electrical signal, wherein the processor is further configured to:
detect a separation between the edge of the mask and the face of the patient by analyzing the electrical signal; and
in response to detecting the separation between the edge of the mask and the face of the patient:
increase an intensity of the first alert; or
cause the display to output a second alert indicating that the mask is separated from the face of the patient.

92. The smart bag-valve mask device of claim 89, the alert being a first alert, the smart bag-valve mask device further comprising:
an accelerometer configured to detect a movement of a chest of the patient, wherein the processor is further configured to:
detect a separation between the mask and the face of the patient by analyzing the movement of the chest of the patient in view of the flow or pressure; and
in response to detecting the separation between the mask and the face of the patient:
increase an intensity of the first alert; or
cause the display to output a second alert indicating that the mask is separated from the face of the patient.

93. A medical device, comprising:
an airway adaptor configured to be connected to an airway of a patient;
a first sensor configured to detect a first parameter of a space fluidly connected to the airway adaptor;
a second sensor configured to detect a second parameter of the space;
a display; and
a processor configured to:
determine a first relationship between the first parameter and the second parameter during a first ventilation;
determine a second relationship between the first parameter and the second parameter during a second ventilation;
determine a discrepancy between the first relationship and the second relationship;
based on determining the discrepancy, identify a leak between the airway adaptor and the airway of the patient;
generate an alert indicating the leak; and
cause the display to output the alert.

94. The medical device of claim 93, wherein the first parameter comprises an air flow rate, and
wherein the second parameter comprises a derivative of an air pressure in the space with respect to time.

95. The medical device of claim 94, wherein the processor is further configured to:
determine a first ventilation volume by integrating the air flow rate over a time in which air flows through the space in a first direction;
determine a second ventilation volume by integrating the air flow rate over a time in which the air flows through the space in a second direction, the first direction being different than the second direction;
wherein the processor is configured to identify the leak further by determining that the first ventilation volume is different than the second ventilation volume.

96. The medical device of claim 93, wherein the first parameter comprises a humidity of the space, and
wherein the second parameter comprises a compression level of an air bag fluidly connected to the space.

97. The medical device of claim 93, further comprising:
a third sensor coupled to a rim of the airway adaptor, the sensor being configured to detect a first value at a first time and a second value at a second time, the first value comprising a first electrical resistance or capacitance, the second value comprising a second electrical resistance or capacitance, the first time occurring when air is traveling into the space, the second time occurring when air is traveling out of the space,
wherein the processor is further configured to:
determine that a difference between the first value and the second value is greater than or equal to a threshold difference, and
wherein the processor is configured to identify the leak further in response to determining that the difference between the first value and the second value is greater than or equal to the threshold difference.

98. The medical device of claim 93, wherein the first parameter comprises an air flow rate,
wherein the second parameter comprises an O₂ level of the space, and
wherein the processor is configured to determine the discrepancy between the first relationship and the second relationship by:
determining that the first relationship is an analogous relationship and determining that the second relationship is not an analogous relationship.

99. The medical device of claim 93, wherein the first parameter comprises a partial pressure of CO₂ in the space, and
wherein the second parameter comprises a partial pressure of O₂ in the space,
wherein the processor is configured to determine the discrepancy between the first relationship and the second relationship by:
determining that the first relationship is a non-inverse relationship and determining that the second relationship is an inverse relationship.

100. A method, comprising:
detecting a first parameter of a space in a bag-valve mask that is fluidly connected to an airway of a patient and that is disposed on a face of the patient;
detecting a second parameter of the space;
identifying a first relationship between the first parameter and the second parameter during a first ventilation;
identifying a second relationship between the first parameter and the second parameter during a second ventilation;
determining a discrepancy between the first relationship and the second relationship;
based on determining the discrepancy between the first relationship and the second relationship, identifying a leak between the bag-valve mask and the face of the patient; and
based on identifying the leak, outputting an alert indicating the leak.

101. The method of claim 100, wherein the first parameter comprises an integral of an air flow rate in the space with respect to time, and
wherein the second parameter comprises an air pressure in the space.

102. The method of claim 100, wherein the first parameter comprises an air flow rate in the space, and
wherein the second parameter comprises a derivative of an air pressure in the space with respect to time.

103. The method of claim 100, wherein the first parameter comprises an air pressure in the space, and
wherein the second parameter comprises a partial pressure of CO₂ or an EtCO₂.

104. The method of claim 100, wherein the first parameter comprises a partial pressure of CO₂ in the space, and
wherein the second parameter comprises a partial pressure of O₂ in the space, a humidity in the space, or a temperature in the space.

105. The method of claim 100, wherein the alert comprises a first alert, the method further comprising:
identifying an electrical resistance between a first electrode and a second electrode attached to a rim of the bag-valve mask; and
in response to determining that the electrical resistance is less than or equal to a threshold, causing a display of the bag-valve mask to output a second alert warning to check for the leak.

106. The method of claim 100, wherein the alert comprises a first alert, the method further comprising:
identifying, via a capacitive sensor attached to a rim of the bag-valve mask, an electrical capacitance; and
in response to determining that the electrical capacitance is less than or equal to a threshold, causing a display of the bag-valve mask to output a second alert warning to check for the leak.

107. The method of claim 100, further comprising:
identifying a blood oxygenation of the patient; and
comparing the blood oxygenation to a threshold,
wherein identifying the leak is further based on comparing the blood oxygenation to the threshold.

108. The method of claim 100, wherein the first relationship comprises a ratio between the first parameter and the second parameter during the first ventilation, and
wherein the second relationship comprises a ratio between the first parameter and the second parameter during the second ventilation.
